(19) United States
(12) Patent Application Publication
(10) Pub. No.: US 2009/0247739 A1
(43) Pub. Date: Oct. 1, 2009

(54) CELLULOSIC SUBSTANCE COMPOSITION, CELLULOSIC SUBSTANCE FILM, OPTICALLY COMPENSATORY FILM, POLARIZING PLATE AND IMAGE DISPLAY DEVICE

(75) Inventors: Yutaka Nozoe, Minami-Ashigara-shi (JP); Tomoko Kuwabara, Minami-Ashigara-shi (JP); Kunihiro Atsumi, Minami-Ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Tokyo (JP)

(21) Appl. No.: 12/411,583

(22) Filed: Mar. 26, 2009

(30) Foreign Application Priority Data
Mar. 26, 2008 (JP) 2008-082211

(51) Int. Cl. C07H 7/02

(52) U.S. Cl. 536/123.1

(57) ABSTRACT

A cellulosic substance composition includes: a cellulosic substance having a branched structure or cyclic structure-containing aliphatic acyl group (A) having from 4 to 30 carbon atoms, the aliphatic acyl group (A) being represented by formula (1):

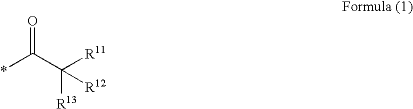

wherein R¹¹, R¹² and R¹³ each independently represents a hydrogen atom or an alkyl group, provided that at least two of R¹¹, R¹² and R¹³ are an alkyl group; R¹¹, R¹² and R¹³ may each independently have a substituent; at least two of R¹¹, R¹² and R¹³ may be connected to each other to form a ring; and a portion of * represents a bond to a cellulose skeleton of the cellulosic substance.

### BACKGROUND OF THE INVENTION

1. Field of the Invention

The present invention relates to a cellulosic substance composition, a cellulosic substance film, a retardation film, an optically compensatory film, an antireflection film, a polarizing plate and a liquid crystal display device. In more detail, the invention relates to a cellulosic substance film having a small photoelastic coefficient and to an optically compensatory film, a polarizing plate and an image display device each using the same.

2. Description of the Related Art

A polarizing plate which is used in liquid crystal display devices and the like includes a polarizer, a first protective film provided on one surface of this polarizer and a second protective film on the other surface of the polarizer. For the respective protective films, a triacetyl cellulose film (TAC film) is widely used in view of excellent transparency and toughness. However, the TAC film is high in its moisture permeability, and therefore, for example, its dimension changes under a high-temperature high-humidity atmosphere due to moisture absorption or the like, thereby generating an optical strain. When the optical strain is generated, deletion or the like is caused in a display screen so the visibility of the display screen is remarkably lowered. When the photoelastic coefficient is large, this optical strain increases, and therefore, a material of the protective film is required to have a small photoelastic coefficient.

There have hitherto been proposed a method of reducing the moisture permeability of a TAC film with an additive (see, for example, JP-A-2005- 113108) and so on. However, it was difficult to avoid the generation of an optical strain in cellulose based resins because there is a limit in reducing the moisture permeability, and a dimensional change is caused not a little under a high-temperature high-humidity atmosphere. Also, there have been proposed a method of using, as a protective film of a polarizing plate, a hydrophobic cycloolefin based resin having a small photoelastic coefficient (see, for example, JP-A-5-212828); a method of using a blend of a cellulose based resin and a cycloolefin based resin (see, for example, JP-A-2007-84800); and so on. However, when the hydrophobic cycloolefin based resin is used, the moisture permeability is too low. Hence, there was involved such a problem that the evaporation of the moisture of an adhesive to be used in sticking to a polarizing plate is slow, thereby generating cloudiness on the polarizing plate or resulting in insufficient adhesion to the polarizing plate.

### SUMMARY OF THE INVENTION

In view of the foregoing problems, an object of the invention is to provide a cellulosic substance film having a small photoelastic coefficient and a retardation film, an optically compensatory film, an antireflection film, a polarizing plate and a liquid crystal display device each using the same.

The foregoing problems of the invention have been attained by the following measures.

(1) A cellulosic substance composition, comprising:

a cellulosic substance having a branched structure or cyclic structure-containing aliphatic acyl group (A) having from 4 to 30 carbon atoms, the aliphatic acyl group (A) being represented by formula (1):

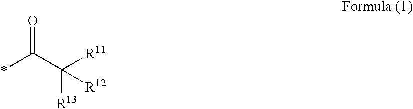

wherein R¹¹, R¹² and R¹³ each independently represents a hydrogen atom or an alkyl group, provided that at least two of R¹¹, R¹² and R¹³ are an alkyl group;

R¹¹, R¹² and R¹³ may each independently have a substituent;

at least two of R¹¹, R¹² and R¹³ may be connected to each other to form a ring; and

a portion of * represents a bond to a cellulose skeleton of the cellulosic substance.

(2) The cellulosic substance composition as described in (1) above,

wherein the aliphatic acyl group (A) is represented by formula (2):

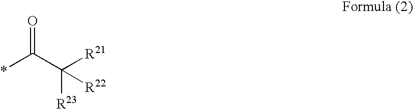

wherein R²¹, R²² and R²³ each independently represents an alkyl group and may each independently have a substituent;

at least two of R²¹, R²² and R²³ may be connected to each other to form a ring; and

a portion of * represents a bond to the cellulose skeleton of the cellulosic substance.

(3) The cellulosic substance composition as described in (1) or (2) above,

wherein the aliphatic acyl group (A) has at least one cyclic structure.

(4) The cellulosic substance composition as described in any of (1) to (3) above,

wherein the aliphatic acyl group (A) has at least two cyclic structures.

(5) The cellulosic substance composition as described in any of (1) to (4) above,

wherein the cellulosic substance is satisfied with expression (1):

0.5≦DSA≦2.0 (I)

wherein DSA represents a degree of substitution of the aliphatic acyl group (A) for substituting a hydrogen atom of a hydroxyl group of cellulose.

(6) The cellulosic substance composition as described in any of (1) to (5) above,

wherein the cellulosic substance further has a linear aliphatic acyl group (B) having from 2 to 4 carbon atoms and is satisfied with expression (II):

2.0≦(DSA+DSB)≦3.0 (II)

wherein DSA represents a degree of substitution of the aliphatic acyl group (A) for substituting a hydrogen atom of a hydroxyl group of cellulose; and

DSB represents a degree of substitution of the aliphatic acyl group (B) for substituting a hydrogen atom of a hydroxyl group of cellulose.

(7) The cellulosic substance composition as described in (6) above,

wherein the aliphatic acyl group (B) is an acetyl group.

(8) A cellulosic substance film, which is formed from the cellulosic substance composition as described in any of (1) to (7) above.

(9) An optically compensatory film, comprising:

the cellulosic substance film as described in (8) above.

(10) A polarizing plate, comprising:

the cellulosic substance film as described in (8) above.

(11) A liquid crystal display device, comprising:

the cellulosic substance film as described in (8) above.

### DETAILED DESCRIPTION OF THE INVENTION

The contents of the invention are hereunder described in detail.

The invention is concerned with a cellulosic substance composition comprising a cellulosic substance having a branched structure or cyclic structure-containing aliphatic acyl group (A) having from 4 to 30 carbon atoms.

In the invention, the terms “having an aliphatic acyl group (A)” mean that in any one of hydroxyl groups which a glucose unit of cellulose has, its oxygen is bound to the foregoing aliphatic acyl group (A).

### <Cellulosic Substance>

In the invention, the branched structure or cyclic structure-containing aliphatic acyl group (A) (the aliphatic acyl group (A) will be hereinafter sometimes referred to as “acyl group (A)”) is an acyl group having from 4 to 30 carbon atoms and may contain an unsaturated bond. The aliphatic acyl group (A) is an acyl group having preferably from 4 to 20 carbon atoms, more preferably from 5 to 18 carbon atoms, and most preferably from 5 to 12 carbon atoms.

In the invention, the acyl group (A) is preferably a group represented by the following formula (1).

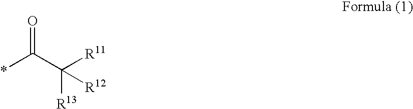

In the formula (1), R¹¹, R¹² and R¹³ each independently represents a hydrogen atom or an alkyl group. However, at least two of R¹¹, R¹² and R¹³ are an alkyl group; and R¹¹, R¹² and R¹³ may each independently have a substituent. At least two of R¹¹, R¹² and R¹³ may be connected to each other to form a ring. A portion of * represents a bond to the cellulose skeleton of the cellulosic substance.

In the invention, the group represented by the formula (1) is bound to an oxygen atom of a hydroxyl group which a glucose unit of cellulose has via a bond represented by *.

Examples of the alkyl group represented by R¹¹ to R¹³ include a methyl group, an ethyl group, a propyl group, an isopropyl group, an n-butyl group, an n-pentyl group, an n-hexyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, a tert-butyl group, a 2-methylbutyl group, an isovaleryl group, a 2-ethylbutyl group, a 2,2-dimethylbutyl group, a 2-methylvaleryl group, a 3-methylvaleryl group, a 4-methylvaleryl group, a 2-propylpentyl group, a 2-methylhexyl group, a 2-ethylhexyl group, a 2-methyl-2-pentyl group, a 2,2-dimethylpentyl group and a 2-octyl group. These groups may be further substituted. Also, when two or more substituents exist, those substituents may be the same or different. If possible, at least two of R¹¹ to R¹³ may be connected to each other to form a ring. Of these, a methyl group, an ethyl group, a propyl group, an n-butyl group, an n-pentyl group, an n-hexyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group and an n-nonadecyl group are preferable.

R¹¹ to R¹³ may be connected to each other to form a ring, and the ring formed may be monocyclic or polycyclic. Examples of the ring which at least two of R¹¹ to R¹³ are connected to each other to form include a cyclopropyl group, a cyclopentyl group and a cyclohexyl group.

Specific examples of the branched structure or cyclic structure-containing aliphatic acyl group (A) include a trimethylacetyl group, a 2-methylbutanoyl group, an isovaleryl group, a 2-ethylbutanoyl group, a 2,2-dimethylbutyryl group, a 2-methylvaleryl group, a 3-methylvaleryl group, a 4-methylvaleryl group, a 2-propylpentanoyl group, a 2-methylhexanoyl group, a 2-ethylhexanoyl group, an isostearoyl group, a tiglinoyl group, a 3,3-dimethylacrylyl group, a 2-methyl-2-pentanoyl group, a 2,2-dimethylpentanoyl group, a citronellyl group, a cyclopropanoyl group, a 1-methylcyclopropanoyl group, a 2-methyl-1-cyclopropanoyl group, a 2,2,3,3-methyl-1-cyclopropanoyl group, a cyclobutanoyl group, a cyclobutylacetyl group, a cyclopentanoyl group, a cyclopentylacetyl group, a cyclopentylpropionyl group, a cyclohexanoyl group, a cyclohexylacetyl group, a cyclohexylpropionyl group, a cyclohexylbutyryl group, a cyclohexylpentanoyl group, a 1-methylcyclohexanoyl group, a dicyclohexylacetyl group, a 1-methyl-1-cyclohexanecarbonyl group, a 1-methylcyclohexenecarbonyl group, a 2-methyl-1-cyclohexanecarbonyl group, a 3-methyl-1-cyclohexanecarbonyl group, a 4-methyl-1-cyclohexanecarbonyl group, a 4-t-butyl-1-cyclohexanecarbonyl group, a 4-pentyl-1-cyclohexanecarbonyl group, a 4-methyl-cyclohexaneacetyl group, a cycloheptanoyl group, a cyclooctanoyl group, a 2-norborneneacetyl group, a 2-methylbicyclo[2,2,1]hept-5-enecarbonyl group, a 4-pentylbicyclo[2,2,2]octane-1-carbonyl group, a bicyclo[3,3,1]nonane-1-carbonyl group, a 3-oxotricyclo[2,2,1,0(2,6)]-heptane-7-carbonyl group, a 3-noradamantanecarbonyl group, a 1-adamantanecarbonyl group, a 1-cyclopentene-1-carbonyl group, a 1-cyclopentene-1-acetyl group, a 1-cyclohexene-1-carbonyl group and a 1-methyl-2-cyclohexene-1-carbonyl group.

The cellulosic substance film formed from the cellulosic substance composition containing a cellulosic substance in which the acyl group (A) is a group represented by the formula (1) is preferable because of a small photoelastic coefficient thereof.

Also, it is preferable that the acyl group (A) is a group represented by the following formula (2).

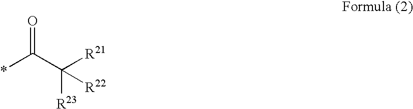

In the formula (2), R²¹, R²² and R²³ each independently represents an alkyl group and may each independently have a substituent. At least two of R²¹, R²² and R²³ may be connected to each other to form a ring. A portion of * represents a bond to the cellulose skeleton of the cellulosic substance.

In the invention, the group represented by the formula (2) is bound to an oxygen atom of a hydroxyl group which a glucose unit of cellulose has via a bond represented by *.

Examples of the alkyl group represented by R²¹ to R²³ include a methyl group, an ethyl group, a propyl group, an isopropyl group, an n-butyl group, an n-pentyl group, an n-hexyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, a tert-butyl group, a 2-methylbutyl group, an isovaleryl group, a 2-ethylbutyl group, a 2,2-dimethylbutyl group, a 2-methylvaleryl group, a 3-methylvaleryl group, a 4-methylvaleryl group, a 2-propylpentyl group, a 2-methylhexyl group, a 2-ethylhexyl group, a 2-methyl-2-pentyl group, a 2,2-dimethylpentyl group and a 2-octyl group. These groups may be further substituted. Also, when two or more substituents exist, those substituents may be the same or different. If possible, at least two of R²¹ to R²³ may be connected to each other to form a ring. Of these, a methyl group, an ethyl group, a propyl group, an n-butyl group, an n-pentyl group, an n-hexyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group and an n-nonadecyl group are preferable.

The cellulosic substance film formed from the cellulosic substance composition containing a cellulosic substance in which the acyl group (A) is a group represented by the formula (2) is preferable because of a small photoelastic coefficient thereof.

The aliphatic acyl group (A) may contain a branched structure or cyclic structure, and preferably contains a cyclic structure. Further, it is particularly preferred to contain at least two cyclic structures.

Specific examples of the branched structure or cyclic structure-containing aliphatic acyl group (A) include a trimethylacetyl group, a 2-methylbutanoyl group, an isovaleryl group, a 2-ethylbutanoyl group, a 2,2-dimethylbutyryl group, a 2-methylvaleryl group, a 3-methylvaleryl group, a 4-methylvaleryl group, a 2-propylpentanoyl group, a 2-methylhexanoyl group, a 2-ethylhexanoyl group, an isostearoyl group, a tiglinoyl group, a 3,3-dimethylacrylyl group, a 2-methyl-2-pentanoyl group, a 2,2-dimethylpentanoyl group, a citronellyl group, a cyclopropanoyl group, a 1-methylcyclopropanoyl group, a 2-methyl-1-cyclopropanoyl group, a 2,2,3,3-methyl-1-cyclopropanoyl group, a cyclobutanoyl group, a cyclobutylacetyl group, a cyclopentanoyl group, a cyclopentylacetyl group, a cyclopentylpropionyl group, a cyclohexanoyl group, a cyclohexylacetyl group, a cyclohexylpropionyl group, a cyclohexylbutyryl group, a cyclohexylpentanoyl group, a 1-methylcyclohexanoyl group, a dicyclohexylacetyl group, a 1-methyl-1-cyclohexanecarbonyl group, a 1-methylcyclohexenecarbonyl group, a 2-methyl-1-cyclohexanecarbonyl group, a 3-methyl-1-cyclohexanecarbonyl group, a 4-methyl-1-cyclohexanecarbonyl group, a 4-t-butyl-1-cyclohexanecarbonyl group, a 4-pentyl-1-cyclohexanecarbonyl group, a 4-methyl-cyclohexaneacetyl group, a cycloheptanoyl group, a cyclooctanoyl group, a 2-norborneneacetyl group, a 2-methylbicyclo[2,2,1]hept-5-enecarbonyl group, a 4-pentylbicyclo[2,2,2]octane-1-carbonyl group, a bicyclo[3,3,1 ]nonane-1-carbonyl group, a 3-oxotricyclo[2,2,1,0(2,6)]-heptane-7-carbonyl group, a 3-noradamantanecarbonyl group, a 1-adamantanecarbonyl group, a 1-cyclopentene-1-carbonyl group, a 1-cyclopentene-1-acetyl group, a 1-cyclohexene-1-carbonyl group and a 1-methyl-2-cyclohexene-1-carbonyl group.

Of these, a trimethylacetyl group, a 2,2-dimethylbutyryl group, a 2,2-dimethylpentanoyl group, a 1-methylcyclopropanoyl group, a 1-methylcyclohexanoyl group, a 1-methylcyclohexenecarbonyl group, a 2-methylbicyclo[2,2,1]hept-5-enecarbonyl group, a bicyclo[3,3,1]nonane-1-carbonyl group and a 1-adamantanecarbonyl group are preferable.

A trimethylacetyl group, a 1-methylcyclopropanoyl group, a 1-methylcyclohexanoyl group, a 2-methylbicyclo[2,2,1]hept-5-ene-carbonyl group and a 1-adamantanecarbonyl group are more preferable.

A 1-adamantanecarbonyl group is the most preferable.

It is preferable that the cellulosic substance composition of the invention contains a cellulosic substance which is satisfied with the following expression (I).

0.5≦DSA≦2.0 (I)

In the expression (I), DSA represents a degree of substitution of the acyl group (A) for substituting a hydrogen atom of a hydroxyl group of cellulose.

It is preferable that the following expression (Ia) is satisfactory; and it is more preferable that the following expression (Ib) is satisfactory.

0.8≦DSA≦2.0 (Ia)

1.2≦DSA≦2.0 (Ib)

What DSA falls within the foregoing range is preferable because the photoelastic coefficient can be reduced, and a suitable optical film can be prepared. When DSA is too large, the brittleness of the film increases, whereas when DSA is too small, the photoelastic coefficient becomes large.

It is preferable that the cellulosic substance of the invention further has an aliphatic acyl group (B) having from 2 to 4 carbon atoms in addition to the foregoing acyl group (A) and contains a cellulosic substance which is satisfied with the following expression (II).

Specific examples of the acyl group (B) include an acetyl group, a propionyl group, a butyryl group and an isobutyryl group. Of these, an acetyl group, a propionyl group and a butyryl group are preferable, with an acetyl group being especially preferable.

2.0≦(DSA+DSB)≦3.0 (II)

In the expression (II), DSA represents a degree of substitution of the aliphatic acyl group (A) for substituting a hydrogen atom of a hydroxyl group of cellulose; and DSB represents a degree of substitution of the aliphatic acyl group (B) having from 2 to 4 carbon atoms for substituting a hydrogen atom of a hydroxyl group of cellulose.

It is preferable that the following expression (IIa) is satisfactory; and it is more preferable that the following expression (IIb) is satisfactory.

2.0<(DSA+DSB)<3.0 (IIa)

2.2<(DSA+DSB)<3.0 (IIb)

What (DSA+DSB) falls within the foregoing range is preferable because the solubility in an organic solvent such as methylene chloride and acetone is enhanced.

Here, DSA and DSB represent a degree of substitution of the foregoing acyl group (A) and the foregoing acyl group (B), respectively. The β-1,4-binding glucose unit which constitutes cellulose has free hydroxyl groups at the 2-position, 3-position and 6-position. In the invention, the “degree of substitution” refers to a proportion in which any one of the hydroxyl groups at the 2-position, 3-position and 6-position is substituted with a specified substituent. When all of the hydroxyl groups at the 2-position, 3-position and 6-position are substituted with a substituent, the degree of substitution is 3.0. Furthermore, in the invention, “(DSA+DSB)” represents a total degree of substitution and represents a degree of substitution of all of the substituents for substituting the hydroxyl groups at the 2-position, 3-position and 6-position. In the invention, the degree of substitution of substituent and the distribution of degree of substitution can be determined by means of ¹³C-NMR by using a method described in Cellulose Communication, 6, 73 to 79 (1999) and Carbohydrate Research, 273, 83 to 91 (1995).

Preferred examples of the cellulosic substance which is satisfied with the expressions (I) and (II) are given in the following Table 1, but it should not be construed that the invention is limited to these specific examples.

TABLE 1

| No. | Substituent A | DSA | Substituent B | DSB | (DSA + DSB) |
|---|---|---|---|---|---|
| A-1 | Trimethylacetyl group | 0.80 | Acetyl group | 2.20 | 3.00 |
| A-3 | 2,2-Dimethylbutyryl group | 0.80 | Acetyl group | 2.20 | 3.00 |
| A-4 | 2,2-Dimethylpentanoyl group | 0.80 | Acetyl group | 2.20 | 3.00 |
| A-5 | Cyclopropanoyl group | 0.80 | Acetyl group | 2.20 | 3.00 |
| A-6 | Cyclobutanoyl group | 0.80 | Acetyl group | 2.20 | 3.00 |
| A-7 | Cyclopentanoyl group | 0.80 | Acetyl group | 2.20 | 3.00 |
| A-8 | Cyclohexanoyl group | 0.80 | Acetyl group | 2.20 | 3.00 |
| A-9 | 1-Methylcyclopropanoyl group | 0.80 | Acetyl group | 2.20 | 3.00 |
| A-10 | 1-Methylcyclohexanoyl group | 0.80 | Acetyl group | 2.20 | 3.00 |
| A-11 | 2-Methylbicyclo[2,2,1]hept-enecarbonyl | 0.80 | Acetyl group | 2.20 | 3.00 |
| | group | | | | |
| A-12 | Bicyclo[3,3,1]nonane-1-carbonyl group | 0.80 | Acetyl group | 2.20 | 3.00 |
| A-13 | 1-Adamantanecarbonyl group | 0.80 | Acetyl group | 2.20 | 3.00 |
| A-14 | Trimethylacetyl group | 0.50 | Acetyl group | 2.50 | 3.00 |
| A-16 | Cyclopropanoyl group | 0.50 | Acetyl group | 2.50 | 3.00 |
| A-17 | 1-Adamantanecarbonyl group | 0.50 | Acetyl group | 2.50 | 3.00 |
| A-18 | Trimethylacetyl group | 1.20 | Acetyl group | 1.80 | 3.00 |
| A-20 | Cyclopropanoyl group | 1.20 | Acetyl group | 1.80 | 3.00 |
| A-21 | 1-Adamantanecarbonyl group | 1.20 | Acetyl group | 1.80 | 3.00 |
| A-22 | Trimethylacetyl group | 1.50 | Acetyl group | 1.50 | 3.00 |
| A-23 | 1-Adamantanecarbonyl group | 1.50 | Acetyl group | 1.50 | 3.00 |
| A-24 | Trimethylacetyl group | 1.80 | Acetyl group | 1.20 | 3.00 |
| A-25 | 1-Adamantanecarbonyl group | 1.80 | Acetyl group | 1.20 | 3.00 |

In the invention, the “cellulosic substance” refers to a compound having a cellulose skeleton obtained by biologically or chemically introducing a functional group into cellulose as a raw material, and a cellulose acylate is preferable.

As a raw material cotton of the cellulose acylate to be used in the invention, not only natural celluloses such as cotton linter and wood pulps (for example, broad-leafed pulps and coniferous pulps) but celluloses having a low degree of polymerization (degree of polymerization: from 100 to 300) obtained by acid hydrolysis of a wood pulp such as microcrystalline cellulose can be used. A mixture thereof may be used as the case may be. These raw material celluloses are described in detail in, for example, Course of Plastic Materials (17): Cellulose Resins, written by Marusawa and Uda and published by The Nikkan Kogyo Shimbun, Ltd. (1970); Journal of Technical Disclosure, No. 2001-1745 (pages 7 to 8) by Japan Institute of Invention and Innovation; and Encyclopedia of Cellulose, page 523, edited by The Cellulose Society of Japan and published by Asakura Publishing Co., Ltd. (2000). These materials can be used, but it should not be construed that the invention is limited thereto.

The viscosity average degree of polymerization of the cellulose acylate is preferably from 300 to 700, more preferably from 350 to 500, and most preferably from 400 to 500. When the average degree of polymerization is not more than 700, the viscosity of a dope solution of the cellulosic substance does not become excessively high, and the film manufacture by means of casting tends to become easy. Also, what the average degree of polymerization is 300 or more is preferable because the strength of the prepared film tends to be more enhanced. The average degree of polymerization can be measured by an intrinsic viscosity method by Uda, et al. (Kazuo Uda and Hideo Saito, Sen'i Gakkaishi (Journal of the Society of Fiber Science and Technology, Japan), Vol. 18, No. 2, pages 105 to 120 (1962)). Specifically, the average degree of polymerization can be measured according to a method described in JP-A-9-95538.

The cellulosic substance to be used in the invention can be obtained by a reaction of cellulose acetate manufactured by Aldrich (degree of acetyl substitution: 2.50) or cellulose acetate manufactured by Daicel Chemical Industries, Ltd. (degree of acetyl substitution: 2.20 (a trade name: L-70) or 1.80 (a trade name: FL-70)) as a starting raw material with a corresponding acid chloride. Also, cellulose acetate having a low degree of acetyl substitution can be properly prepared by using Aldrich's microcrystalline cellulose as a starting raw material by a method described in, for example, Cellulose, pages 283 to 296 (2003) or other methods.

The degree of substitution of the acyl group (A) can be controlled by the temperature and reaction time in the acylation reaction and the kind and amount of the acid chloride or base. The degree of substitution of the acyl group (A) can be adjusted by the kind of cellulose acetate to be used as the starting raw material.

### <Cellulosic Substance Composition>

The cellulosic substance composition of the invention preferably contains 70% by weight or more, more preferably 80% by weight or more, and most preferably 90% by weight or more of the cellulosic substance relative to the whole of the composition.

The cellulosic substance composition of the invention can take various shapes such as a granular shape, a powdered shape, a fibrous shape, a block shape, a solution shape, a melt shape and a film shape.

It is preferable that the raw material in the film manufacture is in a granular or powdered form. Therefore, the cellulose acylate composition after drying may be pulverized or sieved for the purpose of improving the uniformity of particle size or handling properties. When the cellulose acylate composition is in a granular form, it is preferable that 90% by mass or more of particles to be used have a particle size of from 0.5 to 5 mm. (In this specification, mass ratio is equal to weight ratio.) Also, it is preferable that 50% by mass or more of particles to be used have a particle size of from 1 to 4 mm. It is preferable that the cellulose acylate composition particle has a shape close to a sphere as far as possible. Also, the cellulose acylate composition of the invention preferably has an apparent density of from 0.5 to 1.3 g/cm³, more preferably from 0.7 to 1.2 g/cm³, and especially preferably from 0.8 to 1.15 g/cm³. The measurement method of the apparent density is specified in JIS K-7365.

In the invention, the cellulosic substance may be used singly or in admixture of two or more kinds thereof. Also, in addition to the cellulosic substance of the invention, polymer components or various additives can be properly mixed. The component to be mixed is preferably one having excellent compatibility with the cellulosic substance. It is desirable to mix the component such that when formed into a film, the transmittance is preferably 80% or more, more preferably 90% or more, and especially preferably 92% or more.

In the invention, the composition can be prepared by adding various additives which can be generally added in a cellulose acylate (for example, an ultraviolet preventing agent, a plasticizer, an anti-degradation agent, a fine particle and an optical characteristic-adjusting agent) to the cellulosic substance. Also, with respect to the addition timing of an additive to the cellulosic substance, the additive may be added in any step of the preparation process of a dope. Also, such an additive may be added as a preparation step finally in the preparation process of a dope.

Such an additive may be solid or oily. That is, its melting point or boiling point is not particularly limited. For example, a mixture of an ultraviolet absorber of not higher than 20° C. and an ultraviolet absorber of 20° C. or higher can be used, and a mixture of plasticizers can be similarly used. Specifically, a method described in JP-A-2001-151901 can be employed.

### (Stabilizer)

In the invention, in order to keep the stability of the cellulosic substance at the time of high-temperature melt film-forming, it is effective to add a stabilizer. In particular, it is preferred to add at least one member of phenol based stabilizers having a molecular weight of 500 or more and at least one member selected among phosphite ester based stabilizers or thioether based stabilizers having a molecular weight of 500 or more. As a preferred phenol based stabilizer, known arbitrary phenol based stabilizers can be used. Examples of the preferred phenol based stabilizer include hindered phenol based stabilizers. In particular, it is preferable that a substituent exists in a site adjacent to the phenolic hydroxyl group. In that case, the substituent is preferably a substituted or unsubstituted alkyl group having from 1 to 22 carbon atoms, and more preferably a methyl group, an ethyl group, a propyl group, an isopropyl group, a butyl group, an isobutyl group, a t-butyl group, a pentyl group, an isopentyl group, a t-pentyl group, a hexyl group, an octyl group, an isooctyl group or a 2-ethylhexyl group. Also, a stabilizer having a phenol group and a phosphite ester group in the same molecular is exemplified as a preferred material.

These are easily available as commercial products and sold from the following manufactures. These are available from Ciba Specialty Chemicals as IRGANOX 1076, IRGANOX 1010, IRGANOX 3113, IRGANOX 245, IRGANOX 1135, IRGANOX 1330, IRGANOX 259, IRGANOX 565, IRGANOX 1035, IRGANOX 1098 and IRGANOX 1425WL. Also, these are available from Adeka Corporation as ADK STAB AO-50, ADK STAB AO-60, ADK STAB AO-20, ADK STAB AO-70 and ADK STAB AO-80. Furthermore, they are available from Sumitomo Chemical Co., Ltd. as SUMILIZER BP-76, SUMILIZER BP-101 and SUMILIZER GA-80. Also, they are available from Shipro Kasei Kaisha, Ltd. as SEENOX 326M and SEENOX 336B.

Also, it is preferable that a phosphite ester based stabilizer having a molecular weight of 500 or more and having an antioxidant effect is incorporated. Examples of such a compound include compounds described in paragraphs [0023] to [0039] of JP-A-2004-182979 and compounds described in JP-A-51-70316, JP-A-10-306175, JP-A-57-7843 1, JP-A-54-157159 and JP-A-55-13765. Furthermore, as other stabilizers, a stabilizer selected among materials described in detail in Journal of Technical Disclosure, No. 2001-1745, issued Mar. 15, 2001 (pages 17 to 22) by Japan Institute of Invention and Innovation can be used. These are commercially available from Adeka Corporation as ADK STAB 1178, ADK STAB 2112, ADK STAB PEP-8, ADK STAB PEP-24G, ADK STAB PEP-36G and ADK STAB HP-10; and from Clariant as SANDOSTAB F-EPQ.

Also, as the thioether based stabilizer, known arbitrary thioether based stabilizers can be used. These are commercially available from Sumitomo Chemical Co., Ltd. as SUMILIZER TPL, SUMILIZER TPM, SUMILIZER TPS and SUMILIZER TDP. These are also available from Adeka Corporation as ADK STAB AO-412S. In using these stabilizers, at least one member of phenol based stabilizers and at least one member selected among phosphite ester based stabilizers or thioether based stabilizers are preferably incorporated in an amount of from 0.02 to 3% by mass, and especially preferably from 0.05 to 1% by mass, respectively relative to the cellulose acylate. With respect to the contents of the phenol based stabilizer and the phosphite ester based stabilizer or the thioether based stabilizer, though a ratio thereof is not particularly limited, it is preferably from 1/10 to 10/1 (parts by mass), more preferably from 1/5 to 5/1 (parts by mass), further preferably from 1/3 to 3/1 (parts by mass), and especially preferably from 1/3 to 2/1 (parts by mass).

Furthermore, in the invention, a stabilizer having a phenol group and a phosphite ester group in the same molecule is recommended, too. Such a material is described in JP-A-10-273494. Examples of commercially available products include SUMILIZER GP (manufactured by Sumitomo Chemical Co., Ltd.). Furthermore, long-chain aliphatic amines described in JP-A-61-63686, compounds containing a steric hindrance amine group described in JP-A-6-329830, hindered piperidinyl based light stabilizers described in JP-A-7-90270, organic amines described in JP-A-7-278164 and the like are useful. Preferred amine based stabilizers are commercially available from Adeka Corporation as ADK STAB LA-57, ADK STAB LA-52, ADK STAB LA-67, ADK STAB LA-62 and ADK STAB LA-77; and from Ciba Specialty Chemicals as TINUVIN 765 and TINUVIN 144. A ratio of the amine to the phosphite ester is usually from about 0.01 to 25% by mass.

### (Plasticizer)

By adding a plasticizer to the melted cellulose acylate, a crystal melting temperature (Tm) of the cellulose acylate can be decreased. Though the molecular weight of the plasticizer to be used in the invention is not particularly limited, a plasticizer having a high molecular weight is preferable. For example, the molecular weight of the plasticizer is preferably 500 or more, more preferably 550 or more, and further preferably 600 or more. With respect to the kind of the plasticizer, examples thereof include phosphoric esters, alkylphthalylalkyl glycolates, carboxylic acid esters and fatty acid esters of a polyhydric alcohol. With respect to the shape of the plasticizer, the plasticizer may be solid or oily. That is, the plasticizer is not particularly limited with respect to its melting point or boiling point. In the case of performing the melt film-forming, it is especially preferred to use a non-volatile stabilizer.

Examples of the phosphoric ester include triphenyl phosphate, tricresyl phosphate and phenyl diphenyl phosphate.

Examples of the alkylphthalylalkyl glycolate include methylphthalylmethyl glycolate, ethylphthalylethyl glycolate, propylphthalylpropyl glycolate, butylphthalylbutyl glycolate, octylphthalyloctyl glycolate, methylphthalylethyl glycolate, ethylphthalylmethyl glycolate, ethylphthalylpropyl glycolate, methylphthalylbutyl glycolate, ethylphthalylbutyl glycolate, butylphthalylmethyl glycolate, butylphthalylethyl glycolate, propylphthalylbutyl glycolate, butylphthalylpropyl glycolate, methylphthalyloctyl glycolate, ethylphthalyloctyl glycolate, octylphthalylmethyl glycolate and octylphthalylethyl glycolate.

Examples of the carboxylic acid ester include phthalic esters such as dimethyl phthalate, diethyl phthalate, dibutyl phthalate, dioctyl phthalate and diethylhexyl phthalate; and citric esters such as acetyl trimethyl citrate, acetyl triethyl citrate and acetyl tributyl citrate. Besides, it is also preferred to use butyl oleate, methylacetyl linolate, dibutyl sebacate, triacetin, etc. singly or in combination.

The addition amount of such a plasticizer is preferably from 0% by mass to 15% by mass, more preferably from 0% by mass to 10% by mass, and especially preferably from 0% by mass to 8% by mass relative to the cellulose acylate substance to be used for the melt film-forming. Two or more kinds of such a plasticizer may be used in combination as the need arises.

### (Ultraviolet Absorber)

In the cellulosic substance composition to be used for the melt film-forming, an ultraviolet preventing agent may be added. The ultraviolet preventing agent is described in JP-A-60-235852, JP-A-3-199201, JP-A-5-197073, JP-A-5-194789, JP-A-5-271471, JP-A-6-107854, JP-A-6-118233, JP-A-6-148430, JP-A-7-11056, JP-A-7-11055, JP-A-7-11056, JP-A-8-29619, JP-A-8-239509 and JP-A-2000-204173. The addition amount of the ultraviolet preventing agent is preferably from 0.01 to 2% by mass, and more preferably from 0.01 to 1.5% by mass relative to the melt to be prepared.

The following commercial products can be utilized as the ultraviolet absorber. Examples of benzotriazole based ultraviolet absorbers include TINUVIN P (manufactured by Ciba Specialty Chemicals), TINUVIN 234 (manufactured by Ciba Specialty Chemicals), TINUVIN 320 (manufactured by Ciba Specialty Chemicals), TINUVIN 326 (manufactured by Ciba Specialty Chemicals), TINUVIN 327 (manufactured by Ciba Specialty Chemicals), TINUVIN 328 (manufactured by Ciba Specialty Chemicals), SUMISORB 340 (manufactured by Sumitomo Chemical Co., Ltd.) and ADK STAB LA-31 (manufactured by Adeka Corporation). Also, examples of benzophenone based ultraviolet absorbers include SEESORB 100 (manufactured by Shipro Kasei Kaisha, Ltd.), SEESORB 101 (manufactured by Shipro Kasei Kaisha, Ltd.), SEESORB 101S (manufactured by Shipro Kasei Kaisha, Ltd.), SEESORB 102 (manufactured by Shipro Kasei Kaisha, Ltd.), SEESORE 103 (manufactured by Shipro Kasei Kaisha, Ltd.), ADK STAB LA-51 (manufactured by Adeka Corporation), CHEMISORB 111 (manufactured by Chemipro Kasei Kaisha, Ltd.) and UVINUL, D-49 (manufactured by BASF AG). Also, examples of oxalic acid anilide based ultraviolet absorbers include TINUVIN 312 (manufactured by Ciba Specialty Chemicals) and TINUVIN 315 (manufactured by Ciba Specialty Chemicals). Furthermore, examples of salicylic acid based ultraviolet absorbers include SEESORB 201 (manufactured by Shipro Kasei Kaisha, Ltd.) and SEESORB 202 (manufactured by Shipro Kasei Kaisha, Ltd.); and examples of cyano acrylate based ultraviolet absorbers include SEESORB 501 (manufactured by Shipro Kasei Kaisha, Ltd.) and UVINUL N-539 (manufactured by BASF AG).

### (Fine Particle)

In the invention, it is also preferred to add a fine particle in the cellulose acylate composition to be used for the melt film-forming.

In the invention, examples of the fine particle include fine particles of an inorganic compound and fine particles of an organic compound, and either one or both of them may be incorporated. In the invention, an average primary particle size of the fine particle to be incorporated in the cellulosic substance is preferably from 5 nm to 3 μm, more preferably from 5 nm to 2.5 μm, and especially preferably from 20 nm to 2.0 μm. The addition amount of the fine particle is preferably from 0.005 to 1.0% by mass, more preferably from 0.01 to 0.8% by mass, and especially preferably from 0.02 to 0.4% by mass relative to the cellulose acylate. The “average primary particle size” as referred to in the invention means a particle size of the fine particle in a dispersed state (non-coagulated state). The average primary particle size can be measured by known methods such as a dynamic light scattering method (from several nm to 1 μm), a laser diffraction method (from 0.1 μm to several thousand μm) and a laser diffraction and scattering method based on the Mie's theory (from several ten nm to 1 μm).

Preferred examples of the fine particle of an inorganic compound include SiO₂, ZnO, TiO₂, SnO₂, Al₂O₃, ZrO₂, In₂O₃, MgO, BaO, MoO₂, V₂O₅, talc, clay, calcined kaolin, calcined calcium silicate, hydrated calcium silicate, aluminum silicate, magnesium silicate and calcium phosphate. Of these, at least one of SiO₂, ZnO, TiO₂, SnO₂, Al₂O₃, ZrO₂, In₂O₃, MgO, BaO, MoO₂ and V₂O₅ is preferable, with SiO₂, TiO₂, SnO₂, Al₂O₃ and ZrO₂ being more preferable.

As the fine particle of SiO₂, for example, commercially available products such as AEROSIL R972, AEROSIL R972V, AEROSIL R974, AEROSIL R812, AEROSIL 200, AEROSIL 200V, AEROSIL 300, AEROSIL R202, AEROSOL OX50 and AEROSIL TT600 (all of which are manufactured by Nippon Aerosil Co., Ltd.) can be used. Also, as the fine particle of ZrO₂, for example, commercially available products such as AEROSIL R976 and AEROSIL R811 (all of which are manufactured by Nippon Aerosil Co., Ltd.) can be used. Also, SEAHOSTAR KE-E10, SEAHOSTAR E30, SEAHOSTAR E40, SEAHOSTAR E50, SEAHOSTAR E70, SEAHOSTAR E150, SEAHOSTAR W10, SEAHOSTAR W30, SEAHOSTAR W50, SEAHOSTAR P10, SEAHOSTAR P30, SEAHOSTAR P50, SEAHOSTAR P100, SEAHOSTAR P150 and SEAHOSTAR P250 (all of which are manufactured by Nippon Shokubai Co., Ltd.) are useful. Also, SILICA MICROBEAD P-400 and SILICA MICROBEAD 700 (all of which are manufactured by Catalysts & Chemicals Ind. Co., Ltd.) can be utilized. SO-G1, SO-G2, SO-G3, SO-G4, SO-G5, SO-G6, SO-E1, SO-E2, SO-E3, SO-E4, SO-E5, SO-E6, SO-C1, SO-C2, SO-C3, SO-C4, SO-C5 and SO-C6 (all of which are manufactured by Admatechs Co., Ltd.) can be utilized. Furthermore, silica particles manufactured by Moritex Corporation (prepared by converting an aqueous dispersion into a powder) including 8050, 8070, 8100 and 8150 can be utilized.

Preferred examples of the fine particle of an organic compound include polymers such as silicone resins, fluorocarbon resins and acrylic resins, with silicone resins being especially preferable. As the silicone resin, those having a three-dimensional network structure are preferable, and examples thereof include commercially available products having a trade name including TOSPEARL 103, TOSPEARL 105, TOSPEARL 108, TOSPEARL 120, TOSPEARL 145, TOSPEARL 3120 and TOSPEARL 240 (all of which are manufactured by Toshiba Silicone Co., Ltd.).

Furthermore, it is preferable that the fine particle composed of an inorganic compound is subjected to a surface treatment for the purpose of making it stably exist in the cellulosic substance composition and film. It is also preferable that the inorganic fine particle is used after being subjected to a surface treatment. Examples of the surface treatment method include a chemical surface treatment using a coupling agent and a physical surface treatment such as a plasma discharge treatment and a corona discharge treatment. In the invention, the use of a coupling agent is preferable. As the coupling agent, organoalkoxy metal compounds (for example, silane coupling agents and titanium coupling agents) are preferably used. In the case of using an inorganic fine particle as the fine particle (especially, in the case of using SiO₂), a treatment with a silane coupling agent is especially effective. As the silane coupling agent, an organosilane compound can be used. Though the amount of the silane coupling agent to be used is not particularly limited, it is recommended that the silane coupling agent is preferably used in an amount of from 0.005 to 5% by mass, and more preferably from 0.01 to 3% by mass relative to the inorganic fine particle.

The fine particle may be mixed in the cellulosic substance in any step of the film-forming. In the manufacturing process of the cellulose acylate, it is also preferable that the fine particle is added in any step up to reprecipitation, thereby reprecipitating the mixture in a fine particle-containing state.

### (Mold Releasing Agent)

It is also preferable that the cellulosic substance composition to be used for the melt film-forming contains a fluorine atom-containing compound. The fluorine atom-containing compound may be a low-molecular weight compound or a polymer so far as it is able to reveal an action as a mold releasing agent. Examples of the polymer include polymers described in JP-A-2001-269564. The fluorine atom-containing polymer is preferably a polymer obtained by polymerizing a monomer containing, as an essential component, a fluorinated alkyl group-containing ethylenically unsaturated monomer. The fluorinated alkyl group-containing ethylenically unsaturated monomer according to the foregoing polymer is not particularly limited so far as it is a compound having an ethylenically unsaturated group and a fluorinated alkyl group in a molecule thereof. Also, a fluorine atom-containing surfactant can be utilized, and a nonionic surfactant is especially preferable.

### (Pelletization)

It is preferable that the foregoing cellulosic substance and additives are mixed and pelletized prior to the melt film-forming.

With respect to the pelletization, the foregoing cellulosic substance and additives are melted at 150° C. to 250° C. by using a twin-screw or single-screw kneading extruder and extruded into a noodle-shaped form, which is then solidified in water and cut. A pellet can be thus prepared. The pelletization may also be carried out by an underwater cutting method in which the mixture is cut while directly extruding in water. It is more preferable that the pelletization is carried out in vacuo by using a vent type kneading extruder. Furthermore, it is more preferable that the pelletization is carried out while purging the kneading extruder with nitrogen.

The pellet preferably has a size of from 1 mm² to 300 mm² in cross-sectional area and from 1 mm to 30 mm in length, and more preferably from 2 mm² to 100 mm² in cross-sectional area and from 1.5 mm to 10 mm in length.

The number of revolution of the extruder is preferably from 10 rpm to 1,000 rpm, and more preferably from 30 rpm to 500 rpm. The extrusion residence time in the pelletization is from 10 seconds to 30 minutes, and preferably from 30 seconds to 3 minutes.

### [Cellulosic Substance Film]

The invention is also concerned with a cellulosic substance film.

The cellulosic substance film in the invention is a cellulosic substance film formed from the cellulosic substance composition of the invention.

The cellulosic substance film preferably contains 50% by weight or more, more preferably 80% by weight or more, and most preferably 95% by weight or more of the cellulosic substance.

The manufacturing method of the cellulosic substance film of the invention is not particularly limited. However, it is preferable that the cellulosic substance film of the invention is manufactured by a melt film-forming method or a solution film-forming method as described below.

### <Melt Film-Forming>

A preferred embodiment of the case of manufacturing the cellulosic substance film of the invention by a melt film-forming method is described.

It is preferable that the cellulosic substance composition to be used for the melt film-forming has a melt viscosity at 230° C. (melt viscosity at 230° C. of the cellulose acylate film to be manufactured) of from 150 Pa·s to 1,000 Pa·s. Such melt viscosity can be realized by making the composition of substituents fall within the range of the invention and further adjusting the molecular weight. When the molecular weight is too high as compared with the preferred range, there is a concern that the melt viscosity is too high so that the film-forming is hardly achieved. On the other hand, when the molecular weight is too low as compared with the preferred range, there is a concern that not only the strength as a film is too low, but the melt viscosity is too low, whereby a sufficient shear cannot be applied during kneading, and kneading becomes insufficient.

### (Specific Method of Melt Film-Forming)

A specific method of the melt film-forming is hereunder described.

### (1) Drying:

It is preferable that prior to the melt film-forming, the moisture in the pellets is dried. The water content is preferably not more than 0.1% by mass, and more preferably not more than 0.01% by mass.

(2) Melt extrusion:

A dried cellulosic substance resin is supplied into a cylinder from a supply port of an extruder.

A screw compression ratio of the extruder is preferably from 2.5 to 4.5, and more preferably from 3.0 to 4.0. A ratio of L (screw length)/D (screw diameter) is preferably from 20 to 70, and more preferably from 24 to 50. It is preferable that the melt extrusion is carried out at the foregoing temperature.

For the purpose of preventing the oxidation of the resin, it is more preferable that the melt extrusion is carried out in an inert gas (for example, nitrogen) stream within the extruder or while evacuating by using a vented extruder.

### (3) Filtration:

It is preferable that filtration of a breaker plate type is carried out in an outlet of the extruder.

For the purpose of achieving high-precision filtration, a filtration unit of a leaf-type disc filter type is provided after passing through a gear pump. The filtration may be carried out at a single stage or multiple stages.

### (4) Gear Pump:

For the purpose of enhancing the thickness precision (reduction in fluctuation of the discharge amount), it is preferable to set a gear pump between the extruder and a die. Also, for the purpose of stabilizing the extrusion pressure, it is preferable that the fluctuation in temperature of an adaptor for connecting the extruder to the gear pump, the gear pump to the die or the like is made small.

### (5) Die:

All of generally employed T die, fishtail die and hanger coat die are employable so far as the die is designed such that the residence of the melted resin in the die is small. Also, it is preferable that a static mixer is provided just before the T die for the purpose of increasing the uniformity of the resin temperature.

The residence time of the resin after the resin enters the extruder from the supply port until it comes out from the die is preferably from 2 minutes to 60 minutes, and more preferably from 4 minutes to 30 minutes.

### (6) Casting:

The melted resin which has been extruded onto a sheet from the die is cooled for solidification on a casting drum to obtain a film. At that time, it is also preferred to use a touch roll.

The number of the casting drum to be used is preferably from 1 to 8, and more preferably from 2 to 5, and it is preferred to perform gradual cooling. Thereafter, the cooled resin is stripped off from the casting drum and after passing through nip rolls, wound up. The thus obtained unstretched film preferably has a thickness of from 30 μm to 400 μm, and more preferably from 50 μm to 200 μm.

### (7) Winding-Up:

It is preferable that prior to winding up, the both ends are trimmed. A trimmed portion may be reused as a raw material for film. With respect to a wind-up tension, though the film may be wound up at a fixed wind-up tension, it is more preferable that the film is wound up while being tapered corresponding to the wind-up diameter. Also, by adjusting a draw ratio between the nip rolls, the film may be wound up in such a manner that a tension exceeding the specified range is not applied to the film on the way of the line.

A laminated film may be provided on at least one surface of the film prior to winding up.

In the cellulosic substance film of the invention, the amount of the residual organic solvent is preferably not more than 0.03% by mass, more preferably not more than 0.02% by mass, and especially preferably not more than 0.01% by mass. The case where the amount of the residual solvent falls within the foregoing range is preferable because the generation of an odor of the solvent or the change in characteristics of the film to be caused due to the evaporation of the solvent hardly occurs. The melt film-forming method is an effective method for minimizing the residual solvent.

The amount of the residual solvent can be measured by a gas chromatography method or the like.

### <Solution Film-Forming>

Next, a preferred embodiment of the case of manufacturing the cellulosic substance of the invention by a solution film-forming method is described.

In the invention, a solvent of the cellulosic substance is not particularly limited so far as the cellulosic substance can be dissolved therein, casting and film-forming can be performed, and its object can be achieved. Preferred examples of the solvent include chlorine based organic solvents such as dichloromethane, chloroform, 1,2-dichloroethane and tetrachloroethylene; and non-chlorine based organic solvents.

As the non-chlorine based organic solvent to be used in the invention, a solvent selected among esters, ketones and ethers each having from 3 to 12 carbon atoms is preferable. The esters, ketones and ethers may each have a cyclic structure. Compounds having two or more of ester, ketone and ether functional groups (namely, —O—, —CO— and —COO—) can be used as a prime solvent, and these compounds may have other functional group such as an alcoholic hydroxyl group. In the case of a prime solvent having two or more kinds of functional groups, its carbon atom number may fall within the specified range of a compound having any of the functional groups. Examples of the ester having from 3 to 12 carbon atoms include ethyl formate, propyl formate, pentyl formate, methyl acetate, ethyl acetate and pentyl acetate. Examples of the ketone having from 3 to 12 carbon atoms include acetone, methyl ethyl ketone, diethyl ketone, diisobutyl ketone, cyclopentanone, cyclohexanone and methylcyclohexanone. Examples of the ether having from 3 to 12 carbon atoms include diisopropyl ether, dimethoxymethane, dimethoxyethane, 1,4-dioxane, 1,3-dioxolan, tetrahydrofuran, anisole and phenetole. Examples of the organic solvent having two or more kinds of functional groups include 2-ethoxyethyl acetate, 2-methoxyethanol and 2-butoxyethanol.

The chlorine based organic solvent to be used in the invention is not particularly limited so far as the cellulosic substance can be dissolved therein, casting and film-forming can be performed, and its object can be achieved. The chlorine based organic solvent is preferably dichloromethane or chloroform, and especially preferably dichloromethane. Also, there is no particular problem in mixing an organic solvent other than the chlorine based organic solvent. In that case, it is necessary that dichloromethane is used in an amount of at least 50% by mass. The non-chlorine based organic solvent to be used in combination in the invention is hereunder described. That is, as the preferred non-chlorine based organic solvent, a solvent selected among esters, ketones, ethers, alcohols and hydrocarbons each having from 3 to 12 carbon atoms is preferable. The esters, ketones, ethers and alcohols may each have a cyclic structure. Compounds having two or more of ester, ketone and ether functional groups (namely, —O—, —CO— and —COO—) can also be used as the solvent, and these compounds may simultaneously have other functional group such as an alcoholic hydroxyl group. In the case of a solvent having two or more kinds of functional groups, its carbon atom number may fall within the specified range of a compound having any of the functional groups. Examples of the ester having from 3 to 12 carbon atoms include ethyl formate, propyl formate, pentyl formate, methyl acetate, ethyl acetate and pentyl acetate. Examples of the ketone having from 3 to 12 carbon atoms include acetone, methyl ethyl ketone, diethyl ketone, diisobutyl ketone, cyclopentanone, cyclohexanone and methylcyclohexanone. Examples of the ether having from 3 to 12 carbon atoms include diisopropyl ether, dimethoxymethane, dimethoxyethane, 1,4-dioxane, 1,3-dioxolan, tetrahydrofuran, anisole and phenetole. Examples of the organic solvent having two or more kinds of functional groups include 2-ethoxyethyl acetate, 2-methoxyethanol and 2-butoxyethanol.

Also, the alcohol to be used in combination with the chlorine based organic solvent may be preferably linear, branched or cyclic. Above all, the alcohol is preferably a saturated aliphatic hydrocarbon. The hydroxyl group of the alcohol may be any of primary, secondary and tertiary hydroxyl groups. Examples of the alcohol include methanol, ethanol, 1-propanol, 2-propanol, 1-butanol, 2-butanol, tert-butanol, 1-pentanol, 2-methyl-2-butanol and cyclohexanol. Fluorine based alcohols are also useful as the alcohol. Examples thereof include 2-fluoroethanol, 2,2,2-trifluoroethanol and 2,2,3,3-tetrafluoro-1-propanol. Furthermore, the hydrocarbon may be linear, branched or cyclic. All of aromatic hydrocarbons and aliphatic hydrocarbons can be used. The aliphatic hydrocarbon may be saturated or unsaturated. Examples of the hydrocarbon include cyclohexane, hexane, benzene, toluene and xylene.

The non-chlorine based organic solvent to be used in combination with the chlorine based organic solvent as a prime solvent which is used in the foregoing cellulosic substance is not particularly limited and is selected among methyl acetate, ethyl acetate, methyl formate, ethyl formate, acetone, dioxolan, dioxane, ketones or acetoacetic esters having from 4 to 7 carbon atoms and alcohols or hydrocarbons having from 1 to 10 carbon atoms. Preferred examples of the non-chlorine based organic solvent to be used in combination include methyl acetate, acetone, methyl formate, ethyl formate, methyl ethyl ketone, cyclopentanone, cyclohexanone, methyl acetylacetate, methanol, ethanol, 1-propanol, 2-propanol, 1-butanol, 2-butanol, cyclohexanol, cyclohexane and hexane.

The cellulosic substance of the invention is preferably a cellulose acylate solution dissolved in the organic solvent in an amount of from 10 to 35% by mass, more preferably from 13 to 30% by mass, and especially preferably from 15 to 28% by mass. As a method for operating the cellulosic substance in such a concentration, the operation may be performed in a step of dissolution so as to have a prescribed concentration; and after preparing a low-concentration solution (for example, from 9 to 14% by mass) in advance, the solution may be adjusted so as to have a prescribed high concentration in a concentration step as described later. Furthermore, after preparing a high-concentration cellulosic substance solution in advance, the solution may be converted into a prescribed low-concentration cellulosic substance solution by adding various additives. There is no particular problem so far as the operation can be performed so as to have a concentration of the cellulosic substance solution of the invention in any of the foregoing methods.

With respect to the preparation of the cellulosic substance solution (dope) of the invention, its dissolution method is not particularly limited. The dissolution may be carried out at room temperature or is performed by a cooling dissolution method or a high-temperature dissolution method or a combination thereof. With respect to such a dissolution method, the preparation method of a cellulose acylate solution is described in, for example, JP-A-5-163301, JP-A-61-106628, JP-A-58-127737, JP-A-9-95544, JP-A-10-95854, JP-A-10-45950, JP-A-2000-53784, JP-A-11-322946, JP-A-11-322947, JP-A-2-276830, JP-A-2000-273239, JP-A-11-71463, JP-A-04-259511, JP-A-2000-273184, JP-A-11-323017 and JP-A-11-302388. Such a dissolution method of a cellulose acylate in an organic solvent as described in the foregoing patent documents can be properly employed in the invention so far as it falls within the scope of the invention. With respect to the details thereof, especially those of the non-chlorine based solvent system, the dissolution is carried out in a method described in detail in Journal of Technical Disclosure, No. 2001-1745, issued Mar. 15, 2001 (pages 22 to 25) by Japan Institute of Invention and Innovation. Furthermore, in the cellulosic substance solution of the invention, solution concentration and filtration are usually carried out, the details of which are described in Journal of Technical Disclosure, No. 2001-1745, issued Mar. 15, 2001 (page 25) by Japan Institute of Invention and Innovation. In the case of performing the dissolution at a high temperature, in almost all of the cases, the temperature is a boiling point of the organic solvent to be used or higher. In such case, the organic solvent is used in a pressurized state.

It is preferable that the cellulosic substance solution of the invention falls within a certain range as to the viscosity and dynamic storage elastic modulus of the solution. 1 mL of a sample solution was measured using a steel cone (manufactured by TA Instruments) having a diameter of 4 cm/2° for a rheometer (CLS 500, manufactured by TA Instruments). With respect to the measurement condition, the measurement was performed by Oscillation Step/Temperature Ramp in the range of from 40° C. to −10° C. while changing at a rate of 2° C./min, thereby determining a static non-Newtonian viscosity n* at 40° C. (unit: Pa·s) and a storage elastic modulus G′ at −5° C. (unit: Pa). The measurement was started after previously keeping the sample solution at the measurement start temperature so as to have a fixed liquid temperature. In the invention, it is preferable that the viscosity at 40° C. is from 1 to 400 Pa·s and that the dynamic storage elastic modulus at 15° C. is 500 Pa or more; and it is more preferable that the viscosity at 40° C. is from 10 to 200 Pa·s and that the dynamic storage elastic modulus at 15° C. is from 100 to 1,000,000 Pa. Furthermore, it is preferable that the dynamic storage elastic modulus at a low temperature is large as far as possible. For example, in the case where the casting support is at −5° C., it is preferable that the dynamic storage elastic modulus at −5° C. is from 10,000 to 1,000,000 Pa; and in the case where the support is at −50° C., it is preferable that the dynamic storage elastic modulus at −50° C. is from 10,000 to 5,000,000 Pa.

### (Specific Method of Solution Film-Forming)

Next, the manufacturing method of the cellulosic substance of the invention is described. As the method and equipment for manufacturing the cellulose acylate film of the invention, solution casting film-forming methods and solution casting film-forming apparatus which have hitherto been provided for the manufacture of a cellulose acylate film are employed. A dope (cellulosic substance solution) prepared from a dissolver (pot) is once stored in a storage pot, and foams contained in the dope are subjected to defoaming, thereby achieving the final preparation. The dope is sent from a dope discharge port into a pressure die through, for example, a pressure metering gear pump capable of sending a constant amount of the liquid with high precision depending upon the number of revolution; the dope is uniformly cast on a metal support of a casting section running in an endless manner from a nozzle (slit) of the pressure die; and a not fully dried dope layer (also called “web”) is stripped off from the metal support at a stripping point at which the metal support makes substantially a round. The both ends of the obtained web are gripped by clips; the web is conveyed and dried by a tenter while keeping the width; the web is subsequently conveyed by a group of rolls of a drying unit to complete drying; and the dried web is wound up in a prescribed length by a winder. The combination of the tenter and the drying unit composed of a group of rolls varies with its object. In the solution casting film-forming method to be used for silver halide photographic materials and functional protective layers for electronic display, in addition to the solution casting film-forming apparatus, a coating unit is frequently added for the purpose of achieving surface processing of films of a subbing layer, an antistatic layer, an anti-halation layer, a protective layer, etc. These respective manufacturing steps are described in detail in Journal of Technical Disclosure, No. 2001-1745, issued Mar. 15, 2001 (pages 25 to 30) by Japan Institute of Invention and Innovation and are classified into casting (including cocasting), a metal support, drying, stripping, stretching and the like.

Here, in the invention, though a space temperature of the casting section is not particularly limited, it is preferably from −50 to 50° C., more preferably from −30 to 40° C., and especially preferably from −20 to 30° C. In particular, a cellulosic substance solution obtained by casting at a low space temperature is cooled in a moment on the support and increased in gel strength, whereby its organic solvent-containing film can be kept. As a result, it is possible to strip off the film from the support within a short period of time without evaporating the organic solvent from the cellulosic substance. Thus, high-speed casting can be achieved. The measure for cooling the space is not particularly limited, and those using usual air or nitrogen, argon, helium or the like are employable. Also, in that case, the relative humidity is preferably from 0 to 70%, and more preferably from 0 to 50%. Also, in the invention, the temperature of the support in the casting section for casting the cellulosic substance solution is from −50 to 130° C., preferably from −30 to 25° C., and more preferably from −20 to 15° C. In order to keep the casting section at the temperature of the invention, a cooled gas may be introduced into the casting section, or the space may be cooled by disposing a cooling unit in the casting section. At that time, it is important to take care such that water does not attach. Such can be achieved by a method of utilizing a dried gas or the like.

In the invention, with respect to the contents of each layer and casting, the following configuration is especially preferable. That is, a cellulosic substance solution which is characterized in that the cellulosic substance solution is a cellulose acylate solution containing at least one plasticizer which is liquid or solid at 25° C. in an amount of from 0.1 to 20% by mass relative to the cellulose acylate; and/or is a cellulosic substance solution containing at least one liquid or solid ultraviolet absorber in an amount of from 0.001 to 5% by mass relative to the cellulosic substance; and/or is a cellulose acylate solution containing at least one solid fine particle powder having an average particle size of from 5 to 3,000 nm in an amount of from 0.001 to 5% by mass relative to the cellulosic substance; and/or is a cellulose acylate solution containing at least one fluorine based surfactant in an amount of from 0.001 to 2% by mass relative to the cellulose acylate; and/or is a cellulosic substance solution containing at least one stripping agent in an amount of 0.0001 to 2% by mass relative to the cellulose acylate; and/or is a cellulosic substance solution containing at least one anti-degradation agent in an amount of from 0.0001 to 2% by mass relative to the cellulose acylate; and/or a cellulosic substance solution containing at least one optical anisotropy controlling agent in an amount of from 0.1 to 15% by mass relative to the cellulose acylate; and/or is a cellulosic substance solution containing at least one infrared absorber in an amount of from 0.1 to 5% by mass relative to the cellulose acylate, and a cellulosic substance film prepared therefrom are preferable.

In the casting step, one kind of the cellulose acylate solution may be subjected to single-layered casting, or two or more kinds of cellulosic substance solutions may be subjected to simultaneous and/or successive cocasting. In the case of including a casting step of two or more layers, the cellulosic substance solution and the cellulosic substance film to be prepared are a cellulosic substance solution and a cellulosic substance film, respectively, each of which is characterized in that the composition of the chlorine based solvent of each of the layers is either the same or different; that the additive of each of the layers is either one kind or a mixture of two or more kinds; that the addition position of the additive in each of the layers is either the same layer or a different layer; that the concentration of the additive in the solution in each of the layers is either the same or different; that the molecular weight of an associate of each of the layers is either the same or different; that the temperature of the solution of each of the layers is either the same or different; that the coating amount of each of the layers is either the same or different; that the viscosity of each of the layers is either the same or different; that the film thickness after drying of each of the layers is either the same or different; that the materials existing in each of the layers are dispersed in either the same state or a different state; that the physical properties of each of the layers are either the same or different; and that the physical properties of each of the layers are of distribution in physical properties either in a uniform state or a varied state, are preferable. The physical properties as referred to herein include physical properties described in detail in Journal of Technical Disclosure, No. 2001-1745, issued Mar. 15, 2001 (pages 6 to 7) by Japan Institute of Invention and Innovation. Examples thereof include haze, transmittance, spectral characteristics, retardation (Re), retardation (Rth), molecular orientation axis, axis displacement, tear strength, folding strength, tensile strength, difference in Rt of inner and outer windings, creaking, kinetic friction, alkaline hydrolysis, curl value, water content, amount of residual solvent, thermal shrinkage, high-humidity dimensional evaluation, water vapor permeability, planarity of a base, dimensional stability, thermal shrinkage starting temperature, elastic moduhlis and measurement of bright spot foreign matter. Examples thereof further include impedance and surface condition to be used for the evaluation of a base. Also, yellow index, transparency and thermal physical properties (for example, Tg and crystallization heat) as described in detail in Journal of Technical Disclosure, No. 2001-1745, issued Mar. 15, 2001 (page 11) by Japan Institute of Invention and Innovation can be exemplified.

### <Treatment of Cellulosic Substrate Film>

### (Stretching)

It is preferable that the cellulosic substance film of the invention thus manufactured by the melt film-forming method or solution film-forming method is stretched for the purposes of improving the surface condition, revealing Re and Rth, improving a coefficient of linear expansion and the like.

The stretching may be carried out in an on-line manner during the film-forming step or may be carried out in an off-line manner after once winding up after completion of the film-forming. That is, in the case of the melt film-forming, the stretching may be carried out in a stage where cooling during the film-forming has not yet been completed or may be carried out after completion of cooling.

The stretching is preferably carried out at a temperature of from Tg to (Tg+50° C.), more preferably from (Tg+1° C.) to (Tg+30° C.), and especially preferably from (Tg+2° C.) to (Tg+20° C.). A stretch ratio is preferably from 0.1% to 500%, more preferably from 10% to 300%, and especially preferably 30% to 200%. The stretching may be carried out in a single stage or multiple stages. The stretch ratio as referred to herein is one determined according to the following expression.

Stretch ratio (%)=100×{(Length after stretching)−(Length before stretching)}/(Length before stretching)

The stretching is carried out by vertical stretching or lateral stretching or a combination thereof. The vertical stretching can be carried out by, for example, (1) roll stretching (stretching in a longitudinal direction by using two pairs or more of nip rolls in which the peripheral speed on the outlet side is made faster), (2) fixed-end stretching (stretching in a longitudinal direction by gripping the both ends of the film and conveying the film in such a manner that the stretch speed becomes gradually faster toward the longitudinal direction), etc. Furthermore, the lateral stretching can be carried out by, for example, tenter stretching (stretching by gripping the both ends of the film by chucks and widening the film in a lateral direction (perpendicular direction to the longitudinal direction), etc. The vertical stretching and the lateral stretching may be carried out singly (uniaxial stretching) or by a combination thereof (biaxial stretching). In the case of biaxial stretching, the stretching may be successively carried out vertically and laterally (successive stretching) or may be simultaneously carried out (simultaneous stretching).

A stretching rate of vertical stretching and lateral stretching is preferably from 10% to 10,000% per minute, more preferably from 20% to 1,000% per minute, and especially preferably from 30% to 800% per minute. In the case of multi-stage stretching, an average value of stretching rates in the respective stages is indicated.

Subsequent to such stretching, it is preferable that the film is relaxed by from 0% to 10% in the vertical or lateral direction. Furthermore, subsequent to the stretching, it is also preferable that the film is thermally fixed at from 150° C. to 250° C. for from one second to 3 minutes.

The thus stretched film preferably has a thickness of from 10 μm to 300 μm, more preferably from 20 μm to 200 μm, and especially preferably from 30 μm to 100 μm.

Also, it is preferable that an angle θ formed between the film-forming direction (longitudinal direction) and the slow axis of Re of the film is close to 0°, +90° or −90° as far as possible. That is, in the case of vertical direction, it is preferable that the angle 0 is close to 0° as far as possible; and the angle θ is preferably 0±3±, more preferably 0±2°, and especially preferably 0±1°. In the case of lateral stretching, the angle θ is preferably 90±3° or −90±3°, more preferably 90±2° or −90±2°, and especially preferably 90±1° or −90±1°.

In order to suppress light leakage when a polarizing plate is seen from an oblique direction, it is necessary that the transmission axis of a polarization film and the in-plane slow axis of the cellulosic substance film are disposed parallel to each other. The transmission axis of a polarization film in a roll film form as continuously manufactured is in general parallel to a width direction of the roll film. Therefore, in order to continuously stick the foregoing polarization film in a roll film form to a protective film composed of the cellulosic substance film in a roll film form, it is necessary that the in-plane slow axis of the protective film in a roll film form is parallel to the width direction of the film. Accordingly, it is preferable that stretching is performed more chiefly in the width direction. Also, the stretching treatment may be carried out on the way of the film-forming step, or the raw film which has been fabricated and wound up may be subjected to the stretching treatment. In the former case, the stretching may be carried out in a state of containing the residual solvent, and the stretching can be favorably carried out in an amount of the residual solvent of from 2 to 30% by mass.

The thickness of the cellulosic substance film obtained after drying, which is preferably used in the invention, varies depending upon the use purpose. The thickness of the cellulosic substance film is preferably in the range of from 5 to 500 μm, more preferably in the range of from 20 to 300 μm, and further preferably in the range of from 30 to 150 μm. Also, for the use of optical liquid crystal display devices, especially VA liquid crystal display devices, the thickness of the cellulosic substance film is from 40 to 110 μm. The film thickness can be adjusted by adjusting the concentration of solids to be contained in the dope, the slit gap of a nozzle of the die, the extrusion pressure from the die, the speed of the metal support or the like so as to obtain a desired thickness.

The thus obtained cellulosic substance film preferably has a width of from 0.5 to 3 m, more preferably from 0.6 to 2.5 m, and further preferably from 0.8 to 2.2 m. The film is preferably wound up in a length of from 100 to 10,000 m, more preferably from 500 to 7,000 m, and further preferably from 1,000 to 6,000 m per roll. In winding-up, it is preferable to apply knurling to at least one end; the width of knurling is preferably from 3 mm to 50 mm, and more preferably from 5 mm to 30 mm; and the height of knurling is preferably from 0.5 to 500 μm, and more preferably from 1 to 200 μm. This may be single-sided pressing or double-sided pressing.

The foregoing unstretched or stretched cellulosic substance film may be used singly or may be combined with a polarizing plate and used. A liquid crystal layer or a layer having a controlled refractive index (low-reflection layer) or a hard coat layer may be provided and used.

### (Optical Characteristics of Cellulosic Substance Film)

In the invention, Re(λ) and Rth(λ) denote an in-plane retardation and a retardation in the thickness direction, respectively at a wavelength of λ. Re(λ) is measured by making light having a wavelength of λ nm incident in a normal direction of the film by using KOBRA 21ADH (manufactured by Oji Scientific Instruments). Rth(λ) is calculated by KOBRA 21ADH on the basis of retardation values measured in three directions including the foregoing Re(λ), a retardation value measured by making light having a wavelength of λ nm incident in a direction inclined at +40° relative to the normal direction of the film while making an in-plane slow axis (determined by KOBRA 21ADH) as an inclination axis (rotational axis) and a retardation value measured by making light having a wavelength of λ nm incident in a direction inclined at −40° relative to the normal direction of the film while making the in-plane slow axis as an inclination axis (rotational axis); a hypothetical value of an average refractive index; and a film thickness value.

Here, the hypothetical value of an average refractive index can be obtained by referring to Polymer Handbook (John Wiley & Sons, Inc.) or catalogues of various optical films. When an average refractive index value is unknown, it can be measured by using an Abbe's refractometer. Values of the average refractive index of major optical films are exemplified as follows: cellulose acylate (1.48), cycloolefin polymer (1.52), polycarbonate (1.59), polymethyl methacrylate (1.49) and polystyrene (1.59). nx, ny and nz can be calculated by KOBRA21ADH by inputting these hypothetical value of average refractive index and film thickness.

When an oriented film sample is concerned, Re of the cellulosic substance film of the invention is a value obtained by multiplying a value obtained by subtraction of a refractive index in a TD direction (orientation direction) with a refractive index in an MD direction (perpendicular direction to the TD direction) by the thickness (namely, Re=(nx−ny)d). Accordingly, when Re is positive, it is meant that the refractive index (nx) in the TD direction is larger than the refractive index (ny) in the MD direction.

Rth is a value obtained by multiplying a value obtained by subtraction of an average value of a refractive index in a vertical direction and a refractive index in a width direction (an average value of the refractive index in a TD direction and the refractive index in an MD direction in the oriented film as described later) with a refractive index in a thickness direction by the thickness (namely, Rth={(nx+ny)/2−nz}d). Accordingly, when Rth is positive, it is meant that an average value of in-plane refractive index ((nx+ny)/2) is larger than the refractive index (nz) in the thickness direction.

In the cellulosic substance film of the invention, Re and Rth can be adjusted by the total degree of substitution, the distribution of degree of substitution of a substituent at the 2-position, 3-position and 6-position and the stretch ratio.

The scattering of the Re(590) value in the width direction of the film is preferably ±5 nm, and more preferably ±3 nm. Also, the scattering of the Rth(590) value in the width direction of the film is preferably ±10 nm, and more preferably ±5 nm. Also, it is preferable that the scattering of each of the Re value and the Rth value in the length direction of the film falls within the range of the scattering in the width direction.

### (Equilibrium Water Content)

The measurement of a water content can be carried out in the Karl Fischer's method by measuring a sample (7 mm×35 mm) of the cellulose acylate film of the invention by using a coulometric titrator and a sample dryer (AQUACOUNTER AQ-200 and LE-20S, all of which are manufactured by Hiranuma Sangyo Co., Ltd.). The water content is calculated by dividing the amount of water (g) by a sample mass (g).

An equilibrium water content of the cellulose acylate film of the invention at 25° C. and 80% RH is preferably from 0 to 3%, more preferably from 0.1 to 2%, and especially preferably from 0.3 to 1.5%. What the equilibrium water content exceeds 3% is not preferable because when used as a support of an optically compensatory film, the dependency of the retardation by the change in humidity is large so that the optical compensation performance is reduced.

### (Haze)

In the cellulosic substance film of the invention, a value measured using a haze meter (1001DP Model, manufactured by Nippon Denshoku Industries Co., Ltd.) is preferably 0.1 or more and not more than 0.8, more preferably 0.1 or more and not more than 0.7, and especially preferably 0.1 or more and not more than 0.60. By controlling the haze so as to fall within the foregoing range, when the cellulosic substance film of the invention is incorporated as an optically compensatory film into a liquid crystal display device, an image with high contrast is obtainable.

(Photoelastic Modulus) It is preferable that the cellulosic substance film of the invention is used as a protective film of a polarizing plate or a retardation plate. When the cellulosic substance film of the invention is used as a protective film of a polarizing plate or a retardation plate, there may be the case where the birefringence (Re and Rth) is changed due to a stress by elongation or shrinkage by moisture absorption. The change of birefringence following such a stress can be measured as a photoelastic modulus. Its range is preferably from −3×10⁻¹¹ (m²/N) to 3×10⁻¹¹ (m²/N), more preferably from −1×10¹¹ (m²/N) to 1×10⁻¹¹ (m²/N), and especially preferably from −5×10⁻¹² (m²/N) to 5×10⁻¹² (m²/N).

### (Surface Treatment)

The unstretched or stretched cellulosic substance film may be subjected to a surface treatment as the case may be, thereby achieving an enhancement of adhesion between the cellulosic substance film and each of functional layers (for example, an undercoat layer and a backing layer). For example, a glow discharge treatment, an ultraviolet irradiation treatment, a corona treatment, a flame treatment or an acid or alkali treatment can be employed. The glow discharge treatment as referred to herein may be a treatment with a low-temperature plasma occurring under a low-pressure gas of from 10⁻³ to 20 Torr, and furthermore, a treatment with a plasma under an atmospheric pressure is also preferable. A plasma excitation gas means a gas which is plasma-excited under the foregoing condition, and examples thereof include argon, helium, neon, krypton, xenon, nitrogen, carbon dioxide, fluorocarbons such as tetrafluoromethane and mixtures thereof. Such is described in detail in Journal of Technical Disclosure, No. 2001-1745, issued Mar. 15, 2001 (pages 30 to 32) by Japan Institute of Invention and Innovation. For the plasma treatment under an atmospheric pressure, which has recently been watched, for example, irradiation energy of from 20 to 500 Kgy at from 10 to 1,000 Kev is preferably used, and irradiation energy of from 20 to 300 Kgy at from 30 to 500 Kev is more preferably used. Above of all, an alkali saponification treatment is especially preferable, and this is extremely effective as a surface treatment for the cellulosic substance film.

The alkali saponification treatment may be carried out by dipping in a saponification liquid or coating with a saponification liquid. In the case of the dipping method, the treatment can be achieved by passing the film through a tank filled with an aqueous solution at a pH of from 10 to 14, such as NaOH and KOH, and heated at 20° C. to 80° C. for 0.1 minutes to 10 minutes and then neutralizing, washing with water and drying.

In the case of the coating method, a dip coating method, a curtain coating method, an extrusion coating method, a bar coating method and an E type coating method can be employed. It is preferable that a solvent having good wettability for coating the saponification liquid on a transparent support and keeping a surface condition good without causing unevenness on the surface of the transparent support by the solvent of the saponification liquid is chosen as a solvent for the alkali saponification treatment coating solution. Specifically, an alcohol based solvent is preferable, and isopropyl alcohol is especially preferable. Also, an aqueous solution of a surfactant can be used as the solvent. The alkali of the alkali saponification coating solution is preferably an alkali which is soluble in the foregoing solvent, and more preferably KOH or NaOH. The pH of the saponification coating solution is preferably 10 or more, and more preferably 12 or more. With respect to the reaction condition at the time of the alkali saponification, the reaction is preferably carried out at room temperature for from one second to 5 minutes, more preferably from 5 seconds to 5 minutes, and especially preferably from 20 seconds to 3 minutes. After the alkali saponification reaction, it is preferable that the surface coated with the saponification liquid is washed with water or rinsed with an acid and then washed with water. Also, a coating type saponification treatment and provision of an oriented film as described later can be continuously carried out, whereby the number of steps can be reduced. With respect to such a saponification method, for example, the descriptions of JP-A-2002-82226 and WO 02/46809 can be specifically referred to.

For the adhesion to a functional layer, it is preferable that an undercoat layer is provided. This layer may be provided after the foregoing surface treatment or may be provided without performing the surface treatment. The undercoat layer is described in detail in Journal of Technical Disclosure, No. 2001-1745, issued Mar. 15, 2001 (page 32) by Japan Institute of Invention and Innovation.

Such surface treatment and undercoating step can be incorporated at a final stage of the film-forming process, can be carried out singly or can be carried out in a functional layer-application step as described later.

### [Retardation Film]

The cellulosic substance film of the invention can be used as a retardation film.

Also, it is preferable that the cellulosic substance film of the invention is combined with functional layers described in detail in Journal of Technical Disclosure, No. 2001-1745, issued Mar. 15, 2001 (pages 32 to 45) by Japan Institute of Invention and Innovation. Above all, it is preferred to apply a polarization film (formation of a polarizing plate), apply an optically compensatory layer (optically compensatory film) or apply an antireflection layer (antireflection film).

### [Optically Compensatory Film]

The invention is also concerned with an optically compensatory film having an optically anisotropic layer formed by orienting a liquid crystalline compound on the cellulosic substance film or retardation film.

### <Application of Optically Compensatory Layer (Preparation of Optically Compensatory Film)>

The optically anisotropic layer is to compensate a liquid crystal compound in a liquid crystal cell in black displaying of a liquid crystal display device, and the optically compensatory film is prepared by forming an oriented film on the cellulosic substance film and further applying an optically anisotropic layer.

### (Oriented Film)

An oriented film is provided on the foregoing surface-treated cellulosic substance film. This film has a function to regulate the orientation direction of a liquid crystalline molecule. However, when after orienting the liquid crystalline compound, the orientation state is fixed, the oriented film plays its role, and therefore, it is not always essential as a constitutional element. That is, it is also possible to prepare a polarizing plate using the cellulosic substance film of the invention by transferring only the optically anisotropic layer onto the oriented film whose orientation state has been fixed onto a polarizer.

The oriented film can be provided by a measure, for example, a rubbing treatment of an organic compound (preferably a polymer), the oblique vapor deposition of an inorganic compound, the formation of a layer having microgrooves or the accumulation of an organic compound (for example, ω-tricosanic acid, dioctadecylmethylammonium chloride and methyl stearate) by the Langmuir-Blodgett method (LB film). Furthermore, an oriented film in which an orientation function is generated by the application of an electric field, the application of a magnetic field or upon irradiation with light is also known.

It is preferable that the oriented film is formed by a rubbing treatment of a polymer. The polymer to be used for the oriented film basically has a molecular structure having a function to orient a liquid crystalline molecule.

In the invention, in addition to the function to orient a liquid crystalline molecule, it is preferable that a side chain having a crosslinking functional group (for example, a double bond) is bound to the principal chain or that a crosslinking functional group having a function to orient a liquid crystalline molecule is introduced into a side chain.

As the polymer to be used for the oriented film, any of a polymer which is crosslinkable itself or a polymer which is crosslinked with a crosslinking agent can be used, and a plurality of combinations thereof can be used. Examples of the polymer include methacrylate based copolymers described in paragraph [0022] of JP-A-8-338913, styrene based copolymers, polyolefins, polyvinyl alcohol and modified polyvinyl alcohol, poly(N-methylolacrylamide), polyesters, polyimides, vinyl acetate copolymers, carboxymethyl cellulose and polycarbonates. A silane coupling agent can be used as the polymer. Water-soluble polymers (for example, poly(N-methylolacrylamide), carboxymethyl cellulose, gelatin, polyvinyl alcohol and modified polyvinyl alcohol) are preferable; and gelatin, polyvinyl alcohol and modified polyvinyl alcohol are more preferable; and polyvinyl alcohol and modified polyvinyl alcohol are especially preferable. It is especially preferred to use two kinds of polyvinyl alcohol or modified polyvinyl alcohol having a different degree of polymerization in combination. A degree of saponification of polyvinyl alcohol is preferably from 70 to 100%, and more preferably from 80 to 100%. A degree of polymerization of polyvinyl alcohol is preferably from 100 to 5,000.

The side chain having a function to orient a liquid crystalline molecule generally has a hydrophobic group as the functional group. A specific kind of the functional group is determined depending upon the kind of the liquid crystalline molecule and the required orientation state.

A modifying group of the modified polyvinyl alcohol can be introduced by, for example, copolymerization modification, chain transfer modification or block polymerization modification. Examples of the modifying group include hydrophilic groups (for example, a carboxylic group, a sulfonic group, a phosphonic group, an amino group, an ammonium group, an amide group and a thiol group), hydrocarbon groups having from 10 to 100 carbon atoms, fluorine atom-substituted hydrocarbon groups, a thioether group, polymerizable groups (for example, an unsaturated polymerizable group, an epoxy group and an azirinidyl group) and alkoxysilyl groups (for example, a trialkoxy group, a dialkoxy group and a monoalkoxy group). Specific examples of the modified polyvinyl alcohol include those described in paragraphs to [0145] of JP-A-2000-155216 and paragraphs [0018] to [0022] of JP-A-2002-62426.

By binding a side chain having a crosslinking functional group to the principal chain of a polymer for the oriented film or introducing a crosslinking functional group into a side chain having a function to orient a liquid crystalline molecule, it is possible to copolymerize a polymer for the oriented film and a polyfunctional monomer to be incorporated in the optically anisotropic layer. As a result, not only the polyfunctional monomer and the polyfunctional monomer but the polyfunctional monomer and the polymer for the oriented film as well as the polymer for the oriented film and the polymer for the oriented film are firmly bound to each other by covalent bond. Accordingly, by introducing the crosslinking functional group into the polymer for the oriented film, it is possible to remarkably improve the strength of the optically compensatory film.

Likewise the polyfunctional monomer, it is preferable that the crosslinking functional group of the polymer for the oriented film contains a polymerizable group. Specific examples thereof include those described in paragraphs [0080] to [0100] of JP-A-2000-155216. The polymer for the oriented film can be crosslinked with a crosslinking agent separately of the foregoing crosslinking functional group.

Examples of the crosslinking agent include aldehydes, N-methylol compounds, dioxane derivatives, compounds which act upon deactivation of a carboxyl group, active vinyl compounds, active halogen compounds, isooxazole and dialdehyde starch. Two or more kinds of the crosslinking agents may be used in combination. Specific examples thereof include compounds described in paragraphs [0023] to [0024] of JP-A-2002-62426. Aldehydes, especially glutaraldehyde having high reaction activity are preferable.

The addition amount of the crosslinking agent is preferably from 0.1 to 20% by mass, and more preferably from 0.5 to 15% by mass relative to the polymer. It is preferable that the amount of the unreacted crosslinking agent which remains in the oriented film is preferably not more than 1.0% by mass, and more preferably not more than 0.5% by mass. According to this adjustment, the oriented film can be provided with sufficient durability free from reticulation even after it has been used in the liquid crystal display device over a long period of time or it has been allowed to stand under a high-temperature high-humidity atmosphere over a long period of time.

The oriented film can be essentially formed by coating a solution containing the foregoing polymer as an oriented film-forming material and a crosslinking agent on a transparent support, heat drying (crosslinking) and then subjecting to a rubbing treatment. The crosslinking reaction may be effected during any time after coating on the transparent support as described previously. In the case where a water-soluble polymer such as polyvinyl alcohol is used as an oriented film-forming material, it is preferable that the coating solution is a solution in a mixed solvent of an organic solvent having an antifoaming action (for example, methanol) and water. The mixing ratio of water and methanol is preferably from 0/100 to 99/1, and more preferably from 0/100 to 91/9 in terms of a mass ratio. As a result, the generation of foams can be inhibited, and detects on the oriented film and further the layer surface of the optically anisotropic layer are remarkably reduced.

The coating method of the oriented film is preferably a spin coating method, a dip coating method, a curtain coating method, an extrusion coating method, a rod coating method or a roll coating method. Of these, a rod coating method is especially preferable. Also, the thickness of the oriented film after drying is preferably from 0.1 to 10 μm. The heat drying can be generally carried out at from 20° C. to 110° C. In order to form sufficient crosslinking, the heat drying temperature is preferably from 60° C. to 100° C., and more preferably from 80° C. to 100° C. The drying can be usually carried out for a time of from one minute to 36 hours, and preferably from one minute to 30 minutes. It is preferable that the pH is set up at an optimal value for the crosslinking agent to be used. In the case where glutaraldehyde is used, the pH is usually from 4.5 to 5.5, and especially preferably 5.

The oriented film is provided on the transparent support or the foregoing undercoat layer. The oriented film can be obtained by crosslinking the foregoing polymer layer and then subjecting the surface thereof to a rubbing treatment.

As the rubbing treatment, a treatment method which is widely used as a step of orienting a liquid crystal to be carried out in manufacturing a liquid crystal display device can be applied. That is, a method in which the surface of the oriented film is rubbed with paper, gauze, felt, rubber or nylon or polyester fibers in a predetermined direction to attain orientation can be employed. In general, this method is carried out by achieving rubbing several times by using a cloth obtained by uniformly weaving fibers having uniform length and thickness or the like.

In the case where the rubbing is industrially carried out, it is achieved by bringing a rotating rubbing roll into contact with the polarization film-provided film under conveyance. It is preferable that all of circularity, cylindricality and deflection (eccentricity) of the rubbing roll are not more than 30 μm. The wrap angle of the film with respect to the rubbing roll is preferably from 0.1 to 90°. However, as described in JP-A-8-160430, when the film is wound on the rubbing roll over an angle of 360° or more, stable rubbing can be obtained, too. The conveyance rate of the film is preferably from 1 to 100 m/min. It is preferable that an appropriate rubbing angle is chosen within the range of from 0 to 60°. When used in a liquid crystal display device, the rubbing angle is preferably from 40 to 50°, and especially preferably 45°.

It is preferable that the thus obtained oriented film has a thickness in the range of from 0.1 to 10 μm.

### (Optically Compensatory Layer)

Next, a liquid crystalline molecule of the optically anisotropic layer is oriented on the oriented film. Thereafter, the polymer for the oriented film is crosslinked by making the polymer for the oriented film react with a polyfunctional monomer to be incorporated in the optically anisotropic layer or crosslinking the polymer for the oriented film by using a crosslinking agent as the need arises.

The liquid crystalline molecule to be used for the optically anisotropic layer includes a rod-shaped liquid crystalline molecule and a discotic liquid crystalline molecule. The rod-shaped liquid crystalline molecule and the discotic liquid crystalline molecule may be each a high-molecular liquid crystal or a low-molecular liquid crystal. Furthermore, a low-molecular liquid crystal which does not exhibit liquid crystallinity upon being crosslinked is also included.

### (1) Rod-Shaped Liquid Crystalline Molecule:

Examples of the rod-shaped liquid crystalline molecule which is preferably used include azomethines, azoxys, cyanobiphenyls, cyaophenyl esters, benzoic acid esters, cyclohexanecarboxylic acid phenyl esters, cyanophenyl cyclohexanes, cyano-substituted phenylpyrimidines, alkoxy-substituted phenylpyrimidines, phenyldioxanes, tolans and alkenylcyclohexylbenzonitriles.

The rod-shaped liquid crystalline molecule also includes a metal complex. Also, a liquid crystal polymer containing a rod-shaped liquid crystalline molecule in the repeating unit can be used as the rod-shaped liquid crystalline molecule. In other words, the rod-shaped liquid crystalline molecule may be bound to the (liquid crystal) polymer.

For the details of the rod-shaped liquid crystalline molecule, reference can be made to Ekisho no Kagaku (Chemistry of Liquid Crystals), Vol. 22 of Survey of Chemistry, Quarterly, 1994, compiled by The Chemistry society of Japan, Chapters 4, 7 and 11 and Liquid Crystal Device Handbook, 142nd Committee of Japan Society for The Promotion of Science, Chapter 3.

The birefringence of the rod-shaped liquid crystalline molecule is preferably in the range of from 0.001 to 0.7.

It is preferable that the rod-shaped liquid crystalline molecule has a polymerizable group for the purpose of fixing its orientation state. The polymerizable group is preferably a radical polymerizable unsaturated group or a cation polymerizable group. Specific examples thereof include polymerizable groups and polymerizable liquid crystal compounds described in paragraphs [0064] to [0086] of JP-A-2002-62427.

### (2) Discotic Liquid Crystalline Molecule:

Examples of the discotic liquid crystalline molecule include benzene derivatives described in the study report from C. Destrade, et al, Mol. Cryst, Vol. 71, page 111 (1981); torxene derivatives described in the study report from C. Destrade, et al, Mol. Cryst., Vol. 122, page 141 (1985) and Physics Lett, A, Vol. 78, page 82 (1990); cyclohexane derivatives described in the study report from B. Kohne, et al, Angew. Chem., Vol. 96, page 70 (1984); and azacrown based or phenylacetylene based macrocycles described in the study report from J. M. Lehn, et al, J. Chem. Commun., page 1794 (1985) and the study report from J. Zhang, et al, J. Am. Chem. Soc., Vol. 116, page 2655 (1994).

Examples of the discotic liquid crystalline molecule include compounds exhibiting liquid crystallinity, in which a linear alkyl group, an alkoxy group or a substituted benzoyloxy group is radially substituted as a side chain of the mother nucleus on the mother nucleus at the center of the molecule. Such a compound preferably has a molecule or molecular aggregate which is disposed rotation-symmetrically to give predetermined orientation. The compound which is finally contained in the optically anisotropic layer formed of the discotic liquid crystalline molecule is not always a discotic liquid crystalline molecule. For example, a low molecular discotic liquid crystalline molecule having a group which reacts with heat or light, resulting in polymerization or crosslinking due to the reaction with heat or light and becomes a higher polymer having no liquid crystallinity is included. Preferred examples of the discotic liquid crystalline molecule include those described in JP-A-8-50206. Also, the polymerization of the discotic liquid crystalline molecule is described in JP-A-8-27284.

In order to fix the discotic liquid crystalline molecule by polymerization, it is necessary that a polymerizable group is bound as a substituent to a discotic core of the discotic liquid crystalline molecule. The discotic core and the polymerizable group are preferably a compound capable of binding to each other via a connecting group. As a result, the orientation state can be kept even during the polymerization reaction. Examples thereof include those described in paragraphs [0151] to [0168] of JP-A-2000-155216.

In hybrid orientation, an angle formed by the major axis (surface of disc) of the discotic liquid crystalline molecule with respect to the surface of the polarization film increases or decreases with an increase of the distance from the surface of the polarization film in the depth direction of the optically anisotropic layer. It is preferable that the angle decreases with an increase of the distance. Furthermore, the change of the angle can be a continuous increase, a continuous decrease, an intermittent increase, an intermittent decrease, a change including a continuous increase and a continuous decrease or an intermittent change including an increase and a decrease. The intermittent change includes a region in which an angle of inclination does not change on the way in the thickness direction. The angle may include a region in which the angle does not change so far as it increases or decreases as a whole. Furthermore, it is preferable that the angle continuously changes.

The average direction of the major axis of the discotic liquid crystalline molecule on the side of the polarization film can be generally adjusted by selecting a material of the discotic liquid crystalline molecule or the oriented film or by selecting the rubbing treatment method. Also, the direction of the major axis (surface of disc) of the discotic liquid crystalline molecule on the side of the surface (air side) can be generally adjusted by selecting the kind of the discotic liquid crystalline molecule or the additive to be used together with the discotic liquid crystalline molecule. Examples of the additive to be used together with the discotic liquid crystalline molecule include a plasticizer, a surfactant, a polymerizable monomer and a polymer. The degree of change in the orientation direction of the major axis of the major axis can be similarly adjusted by selecting the liquid crystal molecule and the additive.

### (Other Composition of Optically Anisotropic Layer)

The combined use of a plasticizer, a surfactant, a polymerizable monomer and the like together with the foregoing liquid crystalline molecule makes it possible to enhance the uniformity of the coating layer, the strength of the layer, the orientation properties of the liquid crystal molecule and the like. It is preferable that the composition has compatibility with the liquid crystal molecule and is able to give a change of the angle of inclination of the liquid crystal molecule or does not inhibit the orientation.

Examples of the polymerizable monomer include radical polymerizable or cation polymerizable compounds. Of these, polyfunctional radical polymerizable monomers which are copolymerizable with the foregoing polymerizable group-containing liquid crystal compound are preferable. Examples thereof include those described in paragraphs [0018 to [0020] of JP-A-2002-296423. The addition amount of the foregoing compound is generally in the range of from 1% by mass to 50% by mass, and preferably in the range of from 5% by mass to 30% by mass relative to the discotic liquid crystalline molecule.

As the surfactant, there are exemplified conventionally known compounds, and fluorine based compounds are especially preferable. Specific examples thereof include compounds described in paragraphs [0028] to [0056] of JP-A-2001-330725.

It is preferable that the polymer to be used together with the discotic liquid crystalline molecule is able to give a change of the angle of inclination to the discotic liquid crystalline molecule.

Examples of the polymer include cellulose acylates. Preferred examples of the cellulose acylate include those described in paragraph [0178] of JP-A-2000-155216. In order to prevent the inhibition of the orientation of the liquid crystalline molecule, the addition amount of the foregoing polymer is preferably in the range of from 0.1% by mass to 10% by mass, and more preferably in the range of from 0.1% by mass to 8% by mass relative to the liquid crystalline molecule.

The discotic nematic liquid crystal phase-solid phase transition temperature of the discotic liquid crystalline molecule is preferably from 70° C. to 300° C., and more preferably from 70° C. to 170° C.

### (Formation of Optically Anisotropic Layer)

The optically anisotropic layer can be formed by coating a coating solution containing a liquid crystalline molecule and optionally, a polymerization initiator and arbitrary components as described later on the oriented film.

As the solvent to be used for the preparation of the coating solution, an organic solvent is preferably used. Examples of the organic solvent include amides (for example, N,N-dimethylformamide), sulfoxides (for example, dimethyl sulfoxide), heterocyclic compounds (for example, pyridine), hydrocarbons (for example, benzene and hexane), alkyl halides (for example, chloroform, dichloromethane and tetrachloroethane), esters (for example, methyl acetate and butyl acetate), ketones (for example, acetone and methyl ethyl ketone) and ethers (for example, tetrahydrofuran and 1,2-dimethoxyethane). Of these, alkyl halides and ketones are preferable. Two or more kinds of organic solvents may be used in combination.

Coating of the coating solution can be carried out by a known method (for example, a wire bar coating method, an extrusion coating method, a direct gravure coating method, a reverse gravure coating method and a die coating method).

The thickness of the optically anisotropic layer is preferably from 0.1 to 20 μm, more preferably from 0.5 to 15 μm, and especially preferably from 1 to 10 μm.

### (Fixing of Orientation State of Liquid Crystalline Molecule)

The thus oriented liquid crystalline molecule can be fixed while keeping the orientation state. It is preferable that fixing is carried out by a polymerization reaction. Examples of the polymerization reaction include a heat polymerization reaction using a heat polymerization initiator and a photopolymerization reaction using a photopolymerization initiator. Of these, a photopolymerization reaction is preferable.

Examples of the photopolymerization initiator include α-carbonyl compounds (those described in U.S. Pat. Nos. 2,367,661 and 2,367,670), acyloin ethers (those described in U.S. Pat. No. 2,448,828), α-hydrocarbon-substituted aromatic acyloin compounds (those described in U.S. Pat. No. 2,722,512), polynuclear quinone compounds (those described in U.S. Pat. Nos. 3,046,127 and 2,951,758), combinations of a triarylimidazole dimer and p-aminophenylketone (those described in U.S. Pat. No. 3,549,367), acridine and phenazine compounds (those described in JP-A-60-105667 and U.S. Pat. No. 4,239,850) and oxadiazole compounds (those described in U.S. Pat. No. 4,212,970).

The amount of the photopolymerization initiator to be used is preferably in the range of from 0.01 to 20% by mass, and more preferably in the range of from 0.5 to 5% by mass relative to the solids content of the coating solution.

It is preferred to use ultraviolet rays for the irradiation with light for polymerizing a liquid crystalline molecule.

The radiation energy is preferably in the range of from 20 mJ/cm² to 50 J/cm², more preferably in the range of from 20 mJ/cm² to 5,000 mJ/cm², and especially preferably in the range of from 100 mJ/cm² to 800 mJ/cm². Also, in order to accelerate the photopolymerization reaction, the irradiation with light may be carried out under a heating condition.

A protective layer may be provided on the optically anisotropic layer.

(Combination with Polarization Film)

It is also preferred to combine this optically compensatory film with a polarization film. Specifically, the optically anisotropic layer is formed by coating the foregoing coating solution for optically anisotropic layer on the surface of the polarization film. As a result, a thin polarizing plate which shows a small stress ((strain) x (sectional area) x (elastic modulus)) following the dimensional change of the polarization film is formed without using a polymer film between the polarization film and the optically anisotropic layer. By installing the polarizing plate using the cellulosic substance film of the invention in a large-sized liquid crystal display device, an image with high display definition can be displayed without causing a problem such as light leakage.

It is preferable that stretching is performed in such a manner that the angle of inclination formed by the polarization film and the optically compensatory layer is in conformity with an angle formed by transmission axes of two polarizing plates to be stuck on the both sides of a liquid crystal cell which configures LCD and the liquid crystal cell in the vertical or lateral direction. The angle of inclination is usually 45°. However, recently, in transmission type, reflection type or semi-transmission type LCDs, devices in which the angle of inclination is not always 45° have been developed. Thus, it is preferable that the stretching direction can be arbitrarily adjusted in conforming with a design of LCD.

### [Antireflection Film]

The invention is also useful for an antireflection film having an antireflection layer on a cellulosic substance film or a retardation film.

### <Application of Antireflection Layer (Preparation of Antireflection Film)>

In general, the antireflection film is formed by providing a low refractive index layer which also works as an antifouling layer and at least one layer having a refractive index higher than the low refractive index layer (namely, a high refractive index layer and a middle refractive index layer) on a transparent support.

Examples of a method for forming a multilayered film having transparent thin layers made of an inorganic compound (for example, metal oxides) and having a different refractive index from each other include a chemical vapor deposition (CVD) method, a physical vapor deposition (PVD) method and a method in which a colloidal metal oxide particle coat is formed by a sol-gel method of a metal compound such as metal alkoxides and then subjected to a post-treatment (for example, irradiation with ultraviolet rays described in JP-A-9-157855 and a plasma treatment described in JP-A-2002-3273 10) to form a thin layer.

On the other hand, various antireflection films prepared by stacking a thin layer by coating a dispersion having an inorganic particle dispersed in a matrix are proposed as an antireflection film with high productivity. As the antireflection film by coating, there is exemplified an antireflection film prepared by forming a layer having a fine irregular shape on the surface thereof and having antiglare properties imparted thereto on the uppermost layer.

The cellulose acylate film of the invention can be applied to antireflection films to be manufactured by any of the foregoing modes. However, it is especially preferable that the cellulose acylate film of the invention is applied to an antireflection film to be manufactured by a mode by coating (coating type).

### (Layer Configuration of Coating Type Antireflection Film)

An antireflection film composed of a layer configuration in which at least a middle refractive index layer, a high refractive index layer and a low refractive index layer (outermost layer) are stacked in this order on a transparent support is designed so as to have a refractive index which is satisfied with the following relationship.

(Refractive index of high refractive index layer)>(Refractive index of middle refractive index layer)>(Refractive index of transparent support)>(Refractive index of low refractive index layer)

Also, a hard coat layer may be provided between the transparent support and the middle refractive index layer. Furthermore, the configuration may be composed of a middle refractive index hard coat layer, a high refractive index layer and a low refractive index layer. Examples thereof include those described in JP-A-8-122504, JP-A-8-110401, JP-A-10-300902, JP-A-2002-243906 and JP-A-2000-111706.

Also, other function may be imparted to each of the layers. For example, a configuration in which an antifouling low refractive index layer and an antistatic high refractive index layer are stacked (those described in, for example, JP-A-10-206603 and JP-A-2002-243906) is exemplified.

The haze of the antireflection film is preferably not higher than 5%, and more preferably not more than 3%. Also, the strength of the film is preferably H or more, more preferably 2H or more, and especially preferably 3H or more in a pencil hardness test in conformity with JIS K5400.

### (High Refractive Index Layer and Middle Refractive Index Layer)

The high refractive index layer of the antireflection film is composed of a curable film containing at least a high refractive index inorganic compound superfine particle having an average particle size of not more than 100 nm and a matrix binder.

Examples of the high refractive index inorganic compound superfine particle include inorganic compounds having a refractive index of 1.65 or more. Of these, those having a refractive index of 1.9 or more are preferable. Examples thereof include oxides of Ti, Zn, Sb, Sn, Zr, Ce, Ta, La, In, etc. and composite oxides containing such a metal atom.

Examples of a method for obtaining such a superfine particle include a treatment of the particle surface with a surface treating agent (for example, a treatment with a silane coupling agent described in JP-A-11-295503, JP-A-11-153703 and JP-A-2000-9908; and a treatment with an anionic compound or an organometal coupling agent described in JP-A-2001-310432); employment of a core/shell structure in which a high refractive index particle is a core (see, for example, JP-A-2001-166104); and joint use with a specified dispersant (see, for example, JP-A-11-153703, U.S. Pat. No. 6,210,858B1 and JP-A-2002-277609). Examples of a material which forms the matrix include conventionally known thermoplastic resins and curable resin coats.

Furthermore, at least one composition selected from compositions containing a polyfunctional compound having at least two radical polymerizable and/or cation polymerizable groups and compositions containing a hydrolyzable group-containing organometal compound and a partial condensate thereof is preferable. Examples of a compound which is useful for such a composition include compounds described in JP-A-2000-47004, JP-A-2001-315242, JP-A-2001-31871 and JP-A-2001-296401.

Also, a curable film obtained from a colloidal metal oxide obtainable from a hydrolysis condensate of a metal alkoxide and a metal alkoxide composition is preferable. Examples thereof include curable films described in JP-A-2001-293818.

The refractive index of the high refractive index layer is generally from 1.70 to 2.20. The thickness of the high refractive index layer is preferably from 5 nm to 10 μm, and more preferably from 10 nm to 1 μm.

The middle refractive index layer is adjusted so as to have a refractive index which is a value laying between the refractive index of the low refractive index layer and the refractive index of the high refractive index layer. The refractive index of the middle refractive index layer is preferably from 1.50 to 1.70.

### (Low Refractive Index Layer)

The low refractive index layer is formed upon being successively staked on the high refractive index layer. The refractive index of the low refractive index layer is generally from 1.20 to 1.55, and preferably from 1.30 to 1.50.

It is preferable that the low refractive index layer is constructed as an outermost layer having scratch resistance or antifouling properties. As a measure for largely enhancing the scratch resistance, it is effective to impart slipperiness to the surface. Specifically, a conventionally known method for forming a thin layer by introducing a silicone compound or a fluorine-containing compound can be applied.

The refractive index of the fluorine-containing compound is preferably from 1.35 to 1.50, and more preferably from 1.36 to 1.47. Also, the fluorine-containing compound is preferably a compound having a crosslinking or polymerizable functional group containing a fluorine atom in an amount in the range of from 35 to 80% by mass.

Examples thereof include compounds described in paragraphs [0018] to [0026] of JP-A-9-222503, paragraphs [0019] to [0030] of JP-A-1 1-38202, paragraphs [0027] to [0028] of JP-A-2001-40284 and JP-A-2000-284102.

The silicone compound is a compound having a polysiloxane structure and is preferably a compound having a curable functional group or a polymerizable functional group in a polymer chain thereof and having a bridged structure in the film. Examples thereof include reactive silicones (for example, SILAPLANE (manufactured by Chisso Corporation)) and polysiloxanes having a silanol group in both ends thereof (see, for example, JP-A-11-258403).

It is preferable that the crosslinking or polymerization reaction of a fluorine-containing and/or siloxane polymer having a crosslinking or polymerizable group is carried out by coating a coating composition for forming an outermost layer, which contains a polymerization initiator, a sensitizer and the like, and at the same time of or after coating, irradiating light or heating.

Also, a sol-gel cured layer obtained by curing an organometal compound such as silane coupling agents and a silane coupling agent containing a specified fluorine-containing hydrocarbon group in the co-presence of a catalyst is preferable.

Examples of such a sol-gel cured layer include polyfluoroalkyl group-containing silane compounds or partial hydrolysis condensates thereof (for example, compounds described in JP-A-58-142958, JP-A-58-147483, JP-A-58-147484, JP-A-9-157582 and JP-A-11-106704); and silyl compounds having a poly(perfluoroalkyl ether) group which is a fluorine-containing long-chain group (for example, compounds described in JP-A-2000-117902, JP-A-2001-48590 and JP-A-2002-53804).

The low refractive index layer can contain a filler (for example, low refractive index inorganic compounds having an average particle size of primary particle of from 1 to 150 nm, for example, silicon dioxide (silica) and fluorine-containing particles (for example, magnesium fluoride, calcium fluoride and barium fluoride); and organic fine particles described in paragraphs [0020] to [0038] of JP-A-11-3820), a silane coupling agent, a lubricant, a surfactant, etc. as additives other than the foregoing additives.

In the case where the low refractive index layer is disposed beneath the outermost layer, the low refractive index layer may be formed by a vapor phase method (for example, a vacuum vapor deposition method, a sputtering method, an ion plating method and a plasma CVD method). A coating method is preferable because the low refractive index layer can be manufactured at low costs.

The film thickness of the low refractive index layer is preferably from 30 to 200 nm, more preferably from 50 to 150 nm, and especially preferably from 60 to 120 nm.

### (Hard Coat Layer)

The hard coat layer can be provided on the surface of the transparent support for the purpose of imparting a physical strength to the antireflection film. In particular, the hard coat layer is preferably provided between the transparent support and the foregoing high refractive index layer.

The hard coat layer is preferably formed by a crosslinking reaction or polymerization reaction of a photocurable and/or thermally curable compound. The curable functional group is preferably a photopolymerizable functional group; and also, the hydrolyzable functional group-containing organometal compound is preferably an organic alkoxysilyl compound.

Specific examples of such a compound include the same compounds as exemplified in the high refractive index layer.

Specific examples of the composition constituting the hard coat layer include those described in JP-A-2002-144913, JP-A-2000-9908 and WO 00/46617.

The high refractive index layer can also act as the hard coat layer. In that case, it is preferred to form the high refractive index layer by finely dispersing a fine particle and incorporating it into a hard coat layer in the same method as in the high refractive index layer.

The hard coat layer can also act as an antiglare layer (as described later) by incorporating a particle having an average particle size of from 0.2 to 10 μm thereinto to impart an antiglare function.

The thickness of the hard coat layer can be properly designed depending upon applications. The thickness of the hard coat layer is preferably from 0.2 to 10 μm, and more preferably from 0.5 to 7 μm.

The strength of the hard coat layer is preferably H or more, more preferably 2H or more, and especially preferably 3H or more in a pencil hardness test in conformity with JIS K5400. Also, it is preferable that an abrasion amount of a specimen before and after the test is small as far as possible in a taber test according to JIS K5400.

### (Forward Scattering Layer)

In the case of applying to a liquid crystal display device, in order to impart an effect for improving a viewing angle in inclining the viewing angle in the up and down, left and right directions, a forward scattering layer is provided. The forward scattering layer can also act so as to have a hard coat function by dispersing fine particles having a different refractive index from each other in the foregoing hard coat layer.

For example, technologies described in, for example, in JP-A-11-38208 in which a coefficient of forward scattering is specified; JP-2000-199809 in which a relative refractive index of each of a transparent resin and a fine particle is specified; and JP-A-2002-107512 in which a haze value is specified at 40% or more can be employed.

### (Other Layers)

In addition to the foregoing layers, a primer layer, an antistatic layer, an undercoat layer, a protective layer, etc. may be provided.

### (Coating Method)

Each of the layers of the antireflection film can be formed by coating by a dip coating method, an air knife coating method, a curtain coating method, a roll coating method, a wire bar coating method, a gravure coating method, a micro gravure method or an extrusion coating method (as described in U.S. Pat. No. 2,681,294).

### (Antiglare Function)

The antireflection film may have an antiglare function for scattering external light. The antiglare function is obtained by forming irregularities on the surface of the antireflection film. In the case where the antireflection film has an antiglare function, the haze of the antireflection film is preferably from 3 to 30%, more preferably from 5 to 20%, and most preferably from 7 to 20%.

As the method for forming irregularities on the surface of the antireflection film, all of methods capable of sufficiently keeping such a surface shape can be applied. Examples thereof include a method for forming irregularities on the surface of the film by using a fine particle in a low refractive index layer (see, for example, JP-A-2000-271878); a method for forming a layer having an irregular surface by adding a small amount (from 0.1 to 50% by mass) of a relatively large particle (particle size: from 0.05 to 2 μm) in a lower layer (a high refractive index layer, a middle refractive index layer or a hard coat layer) of a low refractive index layer and providing a low refractive index layer thereon while keeping such a shape (see, for example, JP-A-2000-281410, JP-A-2000-95893, JP-A-2001-100004 and JP-A-2001-281407); and a method for physically transferring an irregular shape onto the surface after providing an uppermost layer (antifouling layer) (examples of an embossing method include those described in JP-A-63-278839, JP-A-11-183710 and JP-A-2000-275401).

### [Polarizing Plate]

The polarizing plate of the invention is a polarizing plate comprising a polarization film and two protective films for interposing the polarization film therebetween; and at least one of the two protective films includes at least one of the cellulosic substance film, the retardation film, the optically compensatory film and the antireflection film according to the invention.

### <Polarization Film>

### (Material of Polarization Film)

At present, commercially available polarization films are generally prepared by dipping a stretched polymer in iodine or a dichroic dye solution in a bath to penetrate iodine or the dichroic dye into a binder. As the polarization film, coating type polarization films represented by products of Optiva Inc. can be utilized.

The iodine or dichroic dye in the polarization film exhibits a polarization performance when oriented in the binder. Examples of the dichroic dye to be used include azo based dyes, stilbene based dyes, pyrazolone based dyes, triphenylmethane based dyes, quinoline based dyes, oxazine based dyes, thiazine based dyes and anthraquinone based dyes. It is preferable that the dichroic dye is water-soluble. It is preferable that the dichroic dye has a hydrophilic substituent (for example, sulfo, amino and hydroxyl). Examples thereof include those described in Journal of Technical Disclosure, No. 2001-1745, issued Mar. 15, 2001 (page 58) by Japan Institute of Invention and Innovation.

As the binder of the polarization film, a polymer which is crosslinkable itself or a polymer which can be crosslinked by a crosslinking agent can be used. A plurality of combinations of these polymers can be used. Examples of the binder include methacrylate based copolymers, styrene based copolymers, polyolefins, polyvinyl alcohol, modified polyvinyl alcohol, poly(N-methylolacryamide), polyesters, polyimides, vinyl acetate copolymers, carboxymethyl cellulose and polycarbonates as described in paragraph [0022] of JP-A-8-338913. A silane coupling agent can be used as the polymer.

Of these, water-soluble polymers (for example, poly(N-methylolacrylamide), carboxymethyl cellulose, gelatin, polyvinyl alcohol and modified polyvinyl alcohol) are preferable; gelatin, polyvinyl alcohol and modified polyvinyl alcohol are more preferable; and polyvinyl alcohol and modified polyvinyl alcohol are further preferable. It is especially preferable that two kinds of polyvinyl alcohol or modified polyvinyl alcohol having a different degree of polymerization are used in combination. The degree of saponification of the polyvinyl alcohol is preferably from 70 to 100%, and more preferably from 80 to 100%.

The degree of polymerization of the polyvinyl alcohol is preferably from 100 to 5,000.

With respect to the modified polyvinyl alcohol, reference can be made to JP-A-8-338913, JP-A-9-152509 and JP-A-9-316127. Two or more kinds of such polyvinyl alcohol and modified polyvinyl alcohol may be used in combination.

A lower limit of the binder thickness is preferably 10 μm. From the viewpoint of light leakage of a liquid crystal display device, it is preferable that an upper limit of the binder thickness is thin as far as possible. It is preferably not more than the thickness of current commercially available polarizing plates (about 30 μm), more preferably not more than 25 μm, and especially preferably not more than 20 μm.

The binder of the polarization film may be crosslinked. A crosslinking functional group-containing polymer or monomer may be mixed in the binder, and a crosslinking functional group may be given to the binder polymer itself. The crosslinking can be achieved by light, heat or a change of the pH, and a binder having a crosslinking structure can be formed. The crosslinking agent is described in U.S. Reissue Pat. No. 23,297. Boron compounds (for example, boric acid and borax) can also be used as the crosslinking agent. The addition amount of the crosslinking agent in the binder is preferably from 0.1 to 20% by mass relative to the binder. Orientation properties of a polarizing device and resistance to moist heat of a polarization film become satisfactory.

Even after completion of the crosslinking reaction, the amount of the unreacted crosslinking agent is preferably not more than 1.0% by mass, and more preferably not more than 0.5% by mass. As a result, the weather resistance is enhanced.

### (Stretching of Polarization Film)

It is preferable that after stretching the polarization film (stretching method) or rubbing it (rubbing method), it is dyed with iodine or a dichroic dye.

In the case of the stretching method, a stretch ratio is preferably from 2.5 to 30.0 times, and more preferably from 3.0 to 10.0 times. Stretching can be carried out by dry stretching in air. Also, stretching may be carried out by wet stretching in a state that the polarization film is dipped in water. A stretch ratio of dry stretching is preferably from 2.5 to 5.0 times; and a stretch ratio of wet stretching is preferably from 3.0 to 10.0 times. The stretch ratio as referred to herein denotes {(length of polarization film after stretching)/(length of polarization film before stretching)}. Stretching may be carried out in parallel to the MD direction (parallel stretching) or may be carried out in an oblique direction (oblique stretching). Such stretching may be carried out once or dividedly. By performing the stretching dividedly, the polarization film can be more uniformly stretched at a high stretch ratio. Oblique stretching in which stretching is carried out in an oblique direction by from 10° to 80° is more preferable.

### (a) Parallel Stretching Method:

Prior to stretching, the PVA film is swollen. A degree of swelling is usually from 1.2 to 2.0 times (a ratio of the mass after swelling to the mass before swelling). Thereafter, the swollen PVA film is stretched in an aqueous medium bath or a dyeing bath having a dichroic substance dissolved therein usually at a bath temperature of from 15 to 50° C., and preferably from 17 to 40° C. while continuously conveying it via guide rolls, etc. Stretching can be carried out by gripping by two pairs of nip rolls and making a conveyance rate of the nip rolls of the latter part larger than that of the nip rolls of the former part. In view of the foregoing action and effects, the stretch ratio (a ratio of the length after stretching to the length in the initial state, hereinafter the same) is preferably from 1.2 to 3.5 times, and more preferably from 1.5 to 3.0 times. Thereafter, drying is carried out at from 50° C. to 90° C. to obtain a polarization film.

### (b) Oblique Stretching Method:

As described in JP-A-2002-86554, the oblique stretching method can be carried out by stretching by using a tenter projecting in an oblique direction. Since this stretching is carried out in air, it is necessary that the film is subjected to hydration in advance such that it is easily stretched. The water content is preferably from 5% to 100%, and more preferably from 10% to 100%.

The temperature at the time of stretching is preferably from 40° C. to 90° C., and more preferably from 50° C. to 80° C. The relative humidity is preferably from 50% to 100%, more preferably from 70% to 100%, and especially preferably from 80% to 100%. The advancing rate in the longitudinal direction is preferably 1 m/min or more, and more preferably 3 m/min or more.

After completion of stretching, drying is carried out preferably at from 50° C. to 100° C., and more preferably from 60° C. to 90° C. and preferably for from 0.5 minutes to 10 minutes, and more preferably from one minute to 5 minutes.

The absorption axis of the thus obtained polarization film is preferably from 10° to 80°, more preferably from 30° to 60°, and especially preferably substantially 45° (40° to 50°).

### (Sticking)

The cellulosic substance film after the saponification is stuck with the polarization film fabricated by stretching are stuck to prepare a polarizing plate. As to the sticking direction, it is preferable that the casting axis direction of the cellulosic substance film is formed at 45° relative to the stretching axis direction of the polarizing plate.

An adhesive to be used for sticking is not particularly limited, and examples thereof include PVA based resins (inclusive of modified PVA with an acetoacetyl group, a sulfonic acid group, a carboxyl group, an oxyalkylene group or the like) and boron compound aqueous solutions. Of these, PVA based resins are preferable. The thickness of the adhesive layer after drying is preferably from 0.01 to 10 μm, and especially preferably from 0.05 to 5 μm.

It is preferable that the light transmittance of the thus obtained polarizing plate is high; and it is also preferable that the degree of polarization is high. The transmittance of the polarizing plate is preferably in the range of from 30 to 50%, more preferably in the range of from 35 to 50%, and especially preferably in the range of from 40 to 50% in light having a wavelength of 550 nm. The degree of polarization is preferably in the range of from 90 to 100%, more preferably in the range of from 95 to 100%, and especially preferably in the range of from 99 to 100% in light having a wavelength of 550 nm.

Furthermore, it is possible to prepare a circular polarizing plate by sticking the thus obtained polarizing plate to a λ/4 plate. In that case, sticking is achieved such that the slow axis of the λ/4 plate forms an angle of 45° relative to the absorption axis of the polarizing plate. At that time, the λ/4 plate is not particularly limited, and ones having wavelength dependency such that the shorter the wavelength, the smaller the retardation are more preferable. Furthermore, it is preferable to use a polarization film having an absorption axis inclined at from 20° to 70° relative to the longitudinal direction and a λ/4 plate composed of an optically anisotropic layer made of a liquid crystalline compound.

### [Image Display Device]

The invention is concerned with an image display device including at least one of a cellulosic substance film, a polarizing plate and an optically compensatory film.

As the image display device, a liquid crystal display device can be preferably exemplified.

### <Liquid Crystal Display Device>

The cellulosic substance film of the invention and the polarizing plate, the retardation film and the optical film each using this cellulosic substance film can be each incorporated into a liquid crystal display device. Examples of the liquid crystal display device include TN type, IPS type, FLC type, AFLC type, OCB type, STN type, ECB type, VA type and HAN type display devices. Also, the cellulosic substance film of the invention can be preferably used in any of transmission type, reflection type and semi-transmission type liquid crystal display devices. The respective liquid crystal modes are hereunder described.

### (TN Type Liquid Crystal Display Device)

The cellulosic substance film of the invention may be used as a support of an optically compensatory film of a TN type liquid crystal display device having a liquid crystal cell of a TN mode. The liquid crystal cell of a TN mode and the TN type liquid crystal display device have been well known from old. The optically compensatory film to be used for the TN type liquid crystal display device can be prepared according to methods described in JP-A-3-9325, JP-A-6-148429, JP-A-8-50206 and JP-A-9-26572. Also, it can be prepared according to methods described in the reports of Mori, et al. (Jpn. J Appl. Phys., Vol. 36 (1997), page 143 and Jpn. J Appl. Phys., Vol. 36 (1997), page 1068).

### (STN Type Liquid Crystal Display Device)

The cellulosic substance film of the invention may be used as a support of an optically compensatory film of an STN type liquid crystal display device having a liquid crystal cell of an STN mode. In general, in the STN type liquid crystal display device, the rod-shaped liquid crystalline molecule in the liquid crystal cell is twisted at from 90° to 360°, and the product (And) of a refractive index anisotropy (Δn) and a cell gap (d) of the rod-shaped liquid crystalline molecule is in the range of from 300 to 1,500 nm. The optically compensatory film to be used for the STN type liquid crystal display device can be prepared according to a method described in JP-A-2000-105316.

### (VA Type Liquid Crystal Display Device)

The cellulosic substance film of the invention is especially advantageously used as a support of an optically compensatory film of a VA type liquid crystal display device having a liquid crystal cell of a VA mode. It is preferable that the optically compensatory film to be used in the VA type liquid crystal display device has an Re value of from 0 to 150 nm and an Rth value of from 70 to 400 nm. In the case where two sheets of optically anisotropic polymer films are used for the VA type liquid crystal display device, the Rth value of the films is preferably from 70 to 250 nm. In the case where a single sheet of an optically anisotropic polymer film is used for the VA type liquid crystal display device, the Rth value of the film is preferably from 150 to 400 nm. The VA type liquid crystal display device may be an oriented and divided mode described in, for example, JP-A-10-123576.

### (IPS Type Liquid Crystal Display Device and ECB Type Liquid Crystal Display Device)

The cellulosic substance film of the invention is also favorably used as an optically compensatory film or a protective film of a polarizing plate of an IPS type liquid crystal display device or ECB type liquid crystal display device having a liquid crystal cell of an IPS mode or ECB mode. These modes are an embodiment in which liquid crystal materials are oriented substantially parallel at the time of black displaying, and the liquid crystal molecules are oriented parallel to the substrate surface in a state that no voltage is applied, thereby achieving black displaying. In such an embodiment, the polarizing plate using the cellulosic substance film of the invention contributes to an improvement of tint, an enlargement of viewing angle and an improvement of contrast. In this embodiment, it is preferable that the polarizing plate using the cellulosic substance film of the invention is used on at least one side of the protective films to be disposed between the liquid crystal cell and the polarizing plate (the protective film on the cell side) in the protective films of the polarizing plates of the top and bottom of the liquid crystal cell. It is more preferable that an optically anisotropic layer is disposed between the protective film of the polarizing plate and the liquid crystal cell and that the retardation value of the disposed optically anisotropic layer is set up at not more than 2 times of the Δn·d value of the liquid crystal layer.

### (OCB Type Liquid Crystal Display Device and HAN Type Liquid Crystal Display Device)

The cellulosic substance film of the invention is also advantageously used as a support of an optically compensatory film of an OCB type liquid crystal display device having a liquid crystal cell of an OCB mode or a HAN type liquid crystal display device having a liquid crystal cell of a HAN mode. In the optically compensatory film to be used for the OCB type liquid crystal display device or HAN type liquid crystal display device, it is preferable that a direction in which an absolute value of the retardation value is minimal does not exist in the in-plane and normal directions of the optically compensatory film. The optical properties of the optically compensatory film to be used for the OCB type liquid crystal display device or HAN type liquid crystal display device are determined by optical properties of the optically anisotropic layer, optical properties of the support and disposition between the optically anisotropic layer and the support. The optically compensatory film to be used for the OCB type liquid crystal display device or HAN type liquid crystal display device can be prepared according to a method described in JP-A-9-197397. Also, it can be prepared according to a method described in the report of Mori, et al. (Jpn. J. Appl. Phys., Vol. 38 (1999), page 2837).

### (Reflection Type Liquid Crystal Display Device)

The cellulosic substance film of the invention is also advantageously used as an optically compensatory film of a reflection type liquid crystal display device of a TN type, an STN type, a HAN type or a GH (guest-host) type. These display modes have been well known from old. The TN type reflection type liquid crystal display device can be prepared according to methods described in JP-A-10-123478, WO 98/48320 and Japanese Patent No. 3022477. The optically compensatory film to be used for the reflection type liquid crystal display device can be prepared according to a method described in WO 00/65384.

### (Other Liquid Crystal Display Devices)

The cellulosic substance film of the invention is also advantageously used as a support of an optically compensatory film of an ASM (axially symmetric aligned microcell) type liquid crystal display device having a liquid crystal cell of an ASM mode. The liquid crystal cell of an ASM mode is characterized in that the thickness of the cell is kept by a position-adjustable resin spacer. Other properties are the same as those in the liquid crystal cell of a TN mode. The liquid crystal cell of an ASM mode and the ASM type liquid crystal display device can be prepared according to a method described in the report of Kume, et al. (Kume, et al., SID 98 Digest, 1089 (1998)).

### EXAMPLES

The invention is hereunder described in more detail with reference to the following Examples, but it should not be construed that the invention is limited thereto.

### Synthesis Example 1

### Synthesis of Illustrative Compound A-1

In a 3-L three-necked flask equipped with a mechanical stirrer, a thermometer, a condenser and a dropping funnel, 40 g of diacetyl cellulose having a degree of substitution of 2.20 and 400 mL of pyridine were weighed and taken, and the mixture was stirred at room temperature. 100 mL of trimethylacetyl chloride (manufactured by Aldrich) was gradually added dropwise thereto. After completion of the addition, the mixture was stirred at 70° C. for 5 hours. After completion of the reaction, the reaction solution was allowed to stand for cooling until the temperature returned to room temperature, and the resulting reaction solution was thrown into 10 L of methanol with vigorous stirring. As a result, a white solid was deposited. The white solid was filtered off by means of suction filtration and washed thrice with a large amount of methanol. The obtained white solid was dried at 60° C. overnight and then dried in vacuo at 90° C. for 6 hours to obtain 41 g of desired Illustrative Compound A-1 as a white powder. Mw=270,000, Mw/Mn=2.3, Tg=171° C.

### Synthesis Example 2

### Synthesis of Illustrative Compound A-5

40 g of desired Illustrative Compound A-5 was obtained as a white powder in the same manner as in the foregoing preparation of Illustrative Compound A-1, except for changing the trimethylacetyl chloride to cyclopropanoyl chloride (manufactured by Aldrich). Mw=80,000, Mw/Mn=2.2, Tg=167° C.

### Synthesis Example 3

### Synthesis of Illustrative Compound A-13

45 g of desired Illustrative Compound A-13 was obtained as a white powder in the same manner as in the foregoing preparation of Illustrative Compound A-1, except for changing the trimethylacetyl chloride to 1-adamantanecarbonyl chloride (manufactured by Aldrich). Mw=240,000, Mw/Mn=2.7, Tg=198° C.

### Synthesis Example 4

### Synthesis of Illustrative Compound A-14

40 g of desired Illustrative Compound A-14 was obtained as a white powder in the same manner as in the foregoing preparation of Illustrative Compound A-1, except for using diacetyl cellulose having a degree of substitution of 2.5 in place of the diacetyl cellulose having a degree of substitution of 2.20. Mw=240,000, Mw/Mn=2.4, Tg=168° C.

### Synthesis Example 5

### Synthesis of Illustrative Compound A-18

47 g of desired Illustrative Compound A-18 was obtained as a yellowish white powder in the same manner as in the foregoing preparation of Illustrative Compound A-1, except for using diacetyl cellulose having a degree of substitution of 1.80 in place of the diacetyl cellulose having a degree of substitution of 2.20. Mw=120,000, Mw/Mn=2.2, Tg=177° C.

### Synthesis Example 6

### Synthesis of Illustrative Compound A-21

45 g of desired Illustrative Compound A-21 was obtained as a white powder in the same manner as in the foregoing preparation of Illustrative Compound A-1, except for using diacetyl cellulose having a degree of substitution of 1.80 in place of the diacetyl cellulose having a degree of substitution of 2.20 and using 1-adamantanecarbonyl chloride in place of the trimethylacetyl chloride. Mw=110,000, Mw/Mn=2.2, Tg=203° C.

### Synthesis Example 7

### Synthesis of Illustrative Compound A-23

35 g of desired Illustrative Compound A-23 was obtained as a white powder in the same manner as in the foregoing preparation of Illustrative Compound A-1, except for using diacetyl cellulose having a degree of substitution of 1.50 in place of the diacetyl cellulose having a degree of substitution of 2.20 and using 1-adamantanecarbonyl chloride in place of the trimethylacetyl chloride. Mw=100,000, Mw/Mn=2.2, Tg=208° C.

### Synthesis Example 8

### Synthesis of Illustrative Compound A-24

37 g of desired Illustrative Compound A-24 was obtained as a yellowish white powder in the same manner as in the foregoing preparation of Illustrative Compound A-1, except for using diacetyl cellulose having a degree of substitution of 1.20 in place of the diacetyl cellulose having a degree of substitution of 2.20. Mw=90,000, Mw/Mn=2.4, Tg=180° C.

### Synthesis Example 9

### Synthesis of Comparative Compound B-1

40 g of desired Comparative Compound B-1 was obtained as a white powder in the same manner as in the foregoing preparation of Illustrative Compound A-1, except for using t-butylacetyl chloride in place of the trimethylacetyl chloride. Mw=120,000, Mw/Mn=2.2.

### Synthesis Example 10

### Synthesis of Comparative Compound B-2

In a 3-L three-necked flask equipped with a mechanical stirrer, a thermometer, a condenser and a dropping funnel, 40 g of microcrystalline cellulose and 800 mL of pyridine were weighed and taken, and the mixture was stirred at 60° C. 500 mL of t-butylacetyl chloride (manufactured by Aldrich) was gradually added dropwise thereto. After completion of the addition, the mixture was stirred at 60° C. for 16 hours. After completion of the reaction, the reaction solution was allowed to stand for cooling until the temperature returned to room temperature, and the resulting reaction solution was thrown into 10 L of methanol with vigorous stirring. As a result, a white solid was deposited. The white solid was filtered off by means of suction filtration and washed thrice with a large amount of methanol. The obtained white solid was dried at 60° C. overnight and then dried in vacuo at 90° C. for 6 hours to obtain 80 g of desired Comparative Compound B-2 as a white powder. Mw=110,000, Mw/Mn=2.4, Tg=98° C.

### Synthesis Example 11

### Synthesis of Comparative Compound B-3

43 g of desired Comparative Compound B-3 was obtained as a white powder in the same manner as in the foregoing preparation of Illustrative Compound A-1, except for using valeryl chloride in place of the trimethylacetyl chloride. Mw=230,000, Mw/Mn=2.5.

### Synthesis Example 12

### Synthesis of Comparative Compound B-4

46 g of desired Comparative Compound B-4 was obtained as a white powder in the same manner as in the foregoing preparation of Illustrative Compound A-1, except for using hexanoyl chloride in place of the trimethylacetyl chloride. Mw=220,000, Mw/Mn=2.5.

### Example 1

### <Preparation of Cellulosic Substance Solution>

The following raw materials were charged in a mixing tank and stirred for dissolution while heating, thereby preparing a cellulosic substance solution.

(Composition of cellulosic substance solution)

| | |
|---|---|
| Cellulose acetate having a degree | 100 parts by mass |
| of substitution of 2.93: | |
| Methylene chloride (first solvent): | 402 parts by mass |
| Methanol (second solvent): | 60 parts by mass |

### <Preparation of Cellulosic Substance Film Samples F-1 to F-13

562 parts by mass of the foregoing solution of cellulosic substance composition solution was cast using a glass plate casting machine. A film having a residual solvent amount of 15% by mass was stripped off and framed. Thereafter, the resulting film was heat dried at 100° C. for 10 minutes and further heat dried at 140° C. for 30 minutes, thereby preparing a desired unstretched film sample F-13 (comparison, thickness: 80 μm).

Films F-1 to F-9, F-11 and F-12 were prepared in the same production method, except that in the foregoing preparation of a cellulosic substance solution, the cellulose acetate having a degree of substitution of 2.93 was changed to each of the cellulose esters as shown in the following Table 2. In all of these films, the film thickness is 80 μm. However, a film F-10 was a very brittle film and therefore, could not be subjected to evaluation of optical characteristics.

A part (30 mm×30 mm) of each of the thus obtained film samples was taken out, and with respect to the retardation value, Re against light having a wavelength of 589 nm at 25° C. and 60% RH was measured by KOBRA 21ADH (manufactured by Oji Scientific Instruments). Furthermore, a part (10 mm×110 mm) of each of the thus obtained film samples was taken out, and its photoelastic coefficient was measured using an ellipsometer AEP100 (manufactured by Shimadzu Corporation). The results obtained are shown in the following Table 2.

TABLE 2

| | | | | | | | DSA + | | Photoelastic |
|---|---|---|---|---|---|---|---|---|---|
| | Sample | Compound | Substituent A | DSA | Substituent B | DSB | DSB | Re | coefficient |
| Invention | F-1 | A-1 | Trimethylacetyl group | 0.80 | Acetyl group | 2.20 | 3.00 | 3 | 8 |
| Invention | F-2 | A-5 | Cyclopropanoyl group | 0.80 | Acetyl group | 2.20 | 3.00 | 3 | 7 |
| Invention | F-3 | A-13 | 1-Adamantanecarbonyl | 0.80 | Acetyl group | 2.20 | 3.00 | −1 | 5 |
| | | | group | | | | | | |
| Invention | F-4 | A-14 | Trimethylacetyl group | 0.50 | Acetyl group | 2.50 | 3.00 | 2 | 9 |
| Invention | F-5 | A-18 | Trimethylacetyl group | 1.20 | Acetyl group | 1.80 | 3.00 | 1 | 7 |
| Invention | F-6 | A-21 | 1-Adamantanecarbonyl | 1.20 | Acetyl group | 1.80 | 3.00 | 0 | 2 |
| | | | group | | | | | | |
| Invention | F-7 | A-23 | 1-Adamantanecarbonyl | 1.52 | Acetyl group | 1.50 | 3.02 | −1 | 0 |
| | | | group | | | | | | |
| Invention | F-8 | A-24 | Trimethylacetyl group | 1.80 | Acetyl group | 1.20 | 3.00 | −1 | 1 |
| Comparison | F-9 | B-1 | t-Butylacetyl group | 0.80 | Acetyl group | 2.20 | 3.00 | 3 | 11 |
| Comparison | F-10 | B-2 | t-Butylacetyl group | 3.00 | Acetyl group | 0.00 | 3.00 | — | — |
| Comparison | F-11 | B-3 | Valeryl group | 0.80 | Acetyl group | 2.20 | 3.00 | 2 | 12 |
| Comparison | F-12 | B-4 | Hexanoyl group | 0.80 | Acetyl group | 2.20 | 3.00 | −3 | 13 |
| Comparison | F-13 | TAC | — | 0.00 | Acetyl group | 2.93 | 2.93 | 1 | 15 |

As is clear from the results shown in Table 2, it is noted that all of the unstretched film samples exhibit a small Re value and are optically isotropic. However, it is noted that nevertheless the cellulosic substance film F-13 (comparison) using TAC which is conventionally used as a protective film of a polarizing plate, the cellulosic substance film F-9 (comparison) using, as the substituent A represented by the formula (1), a t-butylacetyl group corresponding to a group in which two of the substituents R¹¹, E¹² and R¹³ are a hydrogen atom and the cellulosic substance films F-11 and F-12 (comparison) each containing, as the substituent A represented by the formula (1), a long-chain aliphatic acyl group have a small Re value, they have a large photoelastic coefficient. On the other hand, the cellulosic substance films F-1 to F-8 of the invention each containing, as the substituent A represented by the formula (1), a branched or cyclic aliphatic acyl group exhibit a very small photoelastic coefficient and hence, are favorable as an optical film.

### Example 2

### Protective Film of a Polarizing Plate

By using the samples of Example 1 (films F-1 to F-8), elliptic polarizing plate samples 001 to 008 were prepared and evaluated in a manner described in Example 1 of JP-A-11-316378. As a result, the optical characteristics of the elliptic polarizing plates obtained from each of the cellulosic substance films of the invention were excellent.

### Example 3

### Liquid Crystal Display Device

By using the samples 001 to 008 of Example 2, polarizing plates with a wide viewing angle described in Example 1 of JP-A-10-48420, optically anisotropic layers containing a discotic liquid crystal molecule and a polyvinyl alcohol-coated oriented film described in Example 1 of JP-A-9-26572, VA type liquid crystal display devices described in FIGS. 2 to 9 of JP-A-2000-154261 and OCB type and HAN type liquid crystal display devices described in FIGS. 10 to 15 of JP-A-2000-154261 were prepared and evaluated. In the display devices obtained by using the cellulosic substance film of the invention, a good performance was obtained in all of the cases.

The cellulosic substance composition of the invention is characterized in that it is able to form a cellulosic substance film having a small photoelastic coefficient. The cellulosic substance film of the invention can be favorably used for a retardation film, an optically compensatory film, an antireflection film, a polarizing plate, an image display device, etc. and can exhibit an excellent display performance.

The entire disclosure of each and every foreign patent application from which the benefit of foreign priority has been claimed in the present application is incorporated herein by reference, as if fully set forth.

What is claimed is:

1. A cellulosic substance composition, comprising:
a cellulosic substance having a branched structure or cyclic structure-containing aliphatic acyl group (A) having from 4 to 30 carbon atoms, the aliphatic acyl group (A) being represented by formula (1): 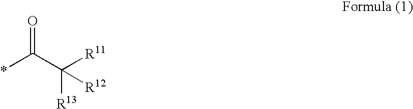
wherein R¹¹, R¹² and R¹³ each independently represents a hydrogen atom or an alkyl group, provided that at least two of R¹¹, R¹² and R¹³ are an alkyl group;
R¹¹, R¹² and R¹³ may each independently have a substituent;
at least two of R¹¹, R¹² and R¹³ may be connected to each other to form a ring; and
a portion of * represents a bond to a cellulose skeleton of the cellulosic substance.

2. The cellulosic substance composition according to claim 1,
wherein the aliphatic acyl group (A) is represented by formula (2): 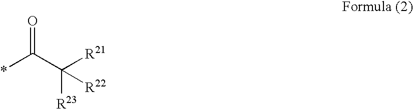
wherein R²¹, R²² and R²³ each independently represents an alkyl group and may each independently have a substituent;
at least two of R²¹, R²² and R²³ may be connected to each other to form a ring; and
a portion of * represents a bond to the cellulose skeleton of the cellulosic substance.

3. The cellulosic substance composition according to claim 1,
wherein the aliphatic acyl group (A) has at least one cyclic structure.

4. The cellulosic substance composition according to claim 1,
wherein the aliphatic acyl group (A) has at least two cyclic structures.

5. The cellulosic substance composition according to claim 1,
wherein the cellulosic substance is satisfied with expression (I):
0.5≦DSA≦2.0 (I)
wherein DSA represents a degree of substitution of the aliphatic acyl group (A) for substituting a hydrogen atom of a hydroxyl group of cellulose.

6. The cellulosic substance composition according to claim 1,
wherein the cellulosic substance further has a linear aliphatic acyl group (B) having from 2 to 4 carbon atoms and is satisfied with expression (II):
2.0≦(DSA+DSB)≦3.0 (II)
wherein DSA represents a degree of substitution of the aliphatic acyl group (A) for substituting a hydrogen atom of a hydroxyl group of cellulose; and
DSB represents a degree of substitution of the aliphatic acyl group (B) for substituting a hydrogen atom of a hydroxyl group of cellulose.

7. The cellulosic substance composition according to claim 6,
wherein the aliphatic acyl group (B) is an acetyl group.

8. A cellulosic substance film, which is formed from the cellulosic substance composition according to claim 1.

9. An optically compensatory film, comprising:
the cellulosic substance film according to claim 8.

10. A polarizing plate, comprising:
the cellulosic substance film according to claim 8.

11. A liquid crystal display device, comprising:
the cellulosic substance film according to claim 8.
